SEQUENCE LISTING (8 sequences)

SEQ ID NO: 1 - protein, 3 aa, Artificial Sequence
  Oligopeptide that can be cleaved selectively by an enzyme
AFK

SEQ ID NO: 2 - protein, 4 aa, Artificial Sequence
  Oligopeptide that can be cleaved selectively by an enzyme
ALAL

SEQ ID NO: 3 - protein, 5 aa, Artificial Sequence
  Oligopeptide that can be cleaved selectively by an enzyme
ALKLL

SEQ ID NO: 4 - protein, 6 aa, Artificial Sequence
  Oligopeptide that can be cleaved selectively by an enzyme
AYGGFL

SEQ ID NO: 5 - protein, 7 aa, Artificial Sequence
  Oligopeptide that can be cleaved selectively by an enzyme
HSSKLQL

SEQ ID NO: 6 - protein, 8 aa, Artificial Sequence
  Oligopeptide that can be cleaved selectively by an enzyme
GPLGIAGQ

SEQ ID NO: 7 - protein, 9 aa, Artificial Sequence
  Oligopeptide that can be cleaved selectively by an enzyme
CDCRGDCFC

SEQ ID NO: 8 - protein, 5 aa, Artificial Sequence
  Oligopeptide that can be cleaved selectively by an enzyme
ALKLL

(19) United States
(12) Patent Application Publication
(10) Pub. No.: US 2006/0281897 A1
(43) Pub. Date: Dec. 14, 2006

(54) Potentialization of the activation of high molecular weight prodrugs

(76) Inventors: Andre Trouet, Herent (BE); Vincent Dubois, Gif-sur-Yvette (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/357,966

(22) Filed: Feb. 22, 2006

Related U.S. Application Data
(63) Continuation-in-part of application No. PCT/FR04/02162, filed on Aug. 19, 2004.
(60) Provisional application No. 60/665,828, filed on Mar. 29, 2005.

(30) Foreign Application Priority Data
Aug. 22, 2003 (FR) FR 0310114

(51) Int. Cl. C07K 14/53; C07K 9/00

(52) U.S. Cl. 530/322; 530/351

(57) ABSTRACT

This invention is directed to a modified form of a prodrug. A typical form of prodrug according to the invention comprises a bulky group, a spacer, a structure that can be cleaved at or near the target cells and a therapeutic agent or a marker, whereby the spacer allows or facilitates the cleavage of the cleavable structure.

### CROSS-REFERENCE TO RELATED APPLICATIONS

This application is a Continuation-in-Part (CIP) of International Application No. PCT/FR2004/002162, filed on Aug. 19, 2004, which claims the benefit of priority to French Patent Application No. FR 03/10114, filed on Aug. 22, 2003, and this application also claims the benefit of priority to U.S. Provisional Patent Application No. 60/665,828, filed on Mar. 29, 2005. The disclosure of each of these applications is incorporated herein by reference in their entireties.

### BACKGROUND OF THE INVENTION

This invention relates to the field of prodrugs, and more particularly the prodrugs that are intended for the treatment and/or diagnosis of cancerous tumors and/or inflammatory reactions.

The prodrugs are pharmacologically inactive molecules that can be transformed in vivo into pharmaceutical agents (e.g., active therapeutic agents) after certain chemical or enzymatic modifications of their structure. The prodrugs allow the release of the pharmaceutical agent, i.e., the transformation of the prodrug into a pharmaceutical agent, at an action site or a target tissue rather than in the circulatory structure or in a non-target tissue. They also make it possible to increase in vivo the therapeutic index (i.e., the activity to toxicity ratio) of therapeutic agents such as the anti-tumor agents like the anthracyclins (such as doxorubicin) and the vinca alkaloids or anti-tumor agents that have anti-inflammatory effects like the methotrexate. Thus, several prodrugs have been developed to date for the purpose of obtaining a high action specificity, a reduced toxicity and an improved stability in the blood and/or the serum.

Prodrugs that exhibit the following basic structure of: therapeutic agent, oligopeptide that can be cleaved by an enzyme that is present in the extracellular environment of target cells, and stabilizing or masking group, have been described in the prior art.

International publication number WO 96/05863 describes in particular a prodrug with formula beta-alanyl-leucyl-alanyl-leucyl-doxorubicin (or else beta-ALA-LEU-ALA-LEU-Dox or beta-ALAL-Dox). This prodrug is stable in the blood (i.e., relatively insensitive to cleavage by peptidases of the blood) and is reactivated in vivo by peptidases that are secreted by a large number of tumor cells. The prodrug is successively hydrolyzed into Ala-Leu-Dox moiety, and then into Leu-Dox moiety in the peritumor extracellular environment. The Leu-Dox penetrates into the cell by diffusion where it is activated by hydrolysis to doxorubicin form (Trouet et al. 2001). The studies of toxicity and activity in vivo with the prodrug above show a reduction in toxicity and an inhibition of the more significant tumor growth relative to the doxorubicin alone. However, the pharmacokinetic studies show that its half-life time for renal elimination is short. The prodrug seems to be eliminated quickly via the urinary tract (Dubois et al. 2002).

International publication number WO 00/33888 proposes the addition to the prodrug beta-Ala-Leu-Ala-Leu-Dox of a group that masks the positive charge of beta-alanine so as to improve its efficacy. This masking group can be, for example, a polyethylene glycol (PEG).

International publication number WO 01/91798 describes prodrugs that have improved stability in the circulatory structure. For example, the prodrugs can be PEGylated, i.e., PEG is used as a stabilizing and/or masking group. The conjugation of this polymer (PEG) brings about an improvement of pharmacokinetic and pharmacodynamic properties of the prodrugs, and through a reduction of the renal elimination, due to the size of the molecule. Actually, the larger the molecule is, the slower its elimination (Harris & Chess, 2003).

Within the framework of research that has led to this invention, the applicant prepared PEGylated prodrugs from a prodrug of doxorubicin (described in International publication number WO 96/05863) by using different sizes of PEG so as to reduce its renal ultrafiltration by the large size of the compound, while keeping its prodrug properties (reactivation by the enzymes that are secreted in the tumor environment). The applicant coupled PEGs of increasing sizes (molecular weights of 350, 750, 2000, 5000, 20,000 and 30,000 Daltons) to the prodrug beta-Ala-Leu-Ala-Leu-Dox and Ala-Leu-Ala-Leu-Dox. To test the reactivation of the PEGylated derivatives of this prodrug, cleavage tests were carried out in vitro. The object of these tests was to evaluate the reactivation of PEGylated derivatives by enzymes secreted by the tumor cells (LS-174T and MCF-7/6) relative to the beta-Ala-Leu-Ala-Leu-Dox that is hydrolyzed in Leu-Dox in the presence of a cancerous cells-conditioned medium. The results of these tests showed the following: 1) that regardless of the size of the PEG, the reactivation of the drug by cleavage of the peptide sequence (Ala-Leu-Ala-Leu) is less effective, compared to the prodrug without the PEG group, and; 2) a correlation between the PEG size and the cleavage of the PEGylated prodrug, that the larger the coupled PEG was, the less the prodrug was cleaved by the enzymes present in the extracellular environment of the target cells. The applicant conceived the non-limiting hypothesis that the reduction of the cleavage of the peptide bond of the prodrug was certainly due to a steric hindrance phenomenon of the PEG. In other words, the more the prodrug comprises a stabilizing or masking group of high molecular weight, the less it is reactivated, which goes against the object sought for a prodrug that has a long half-life.

### BRIEF SUMMARY OF THE INVENTION

This invention is directed to a new prodrug structure to eliminate this steric hindrance phenomenon and to make possible or to facilitate the cleavage of the oligopeptide when the masking and/or stabilizing group is large, while keeping a high specificity of action, a low toxicity, and a stability in the blood and/or serum, preferably in a mammal. In a preferred embodiment, the mammal is a human.

This object is attained by inserting a “molecular arm” or a “molecular spacer” between the masking and/or stabilizing group (for example the PEG), and the peptide sequence that can be cleaved by a “specific” enzyme of this sequence.

The molecular spacers according to the invention, also referred to below as “spacers,” were selected by taking into consideration hydrophilic properties of the units that constitute the spacer.

This invention therefore first has as its object a compound of formula (A)ₚ-(E-B)ₙ-(I)ₘ:

in which:
I is an active substance of interest against target cells,
A is a group that increases the half-life time of B-I in the blood circulation,
E-B is a group connecting A and I, where:
B is a structure that can be cleaved selectively by an enzyme that is present only or preferably close to or at said target cells,
E is a hydrophilic spacer group, stable in the circulatory structure, which separates A from B so as to make possible or to facilitate the cleavage of B close to or at said target cells and thus to make possible or facilitate the release of I or the release of I with a radical or fragment of B,
n is an integer between 1 and either the total number of reactive functions of I on which connecting groups E-B can be coupled, or the total number of reactive functions of A on which connecting groups E-B can be coupled,
m is an integer between 1 and the total number of reactive functions of A on which connecting groups E-B can be coupled, or the total number of reactive functions of B on which I can be coupled,
p is an integer between 1 and the total number of reactive functions of I, on which connecting groups E-B can be coupled, or the total number of reactive functions of E on which A can be coupled,

with, optionally, when p=1, n=m, and when m=1, n=p.

### BRIEF DESCRIPTION OF THE FIGURES

FIG. 1 represents diagrammatically the two methods for synthesis of PEGylated prodrugs of doxorubicin.

FIG. 2 shows the results of cytotoxicity texts of doxorubicin, beta-ALAL-Dox, PEG₂₀₀₀-beta-ALAL-Dox, PEG₂₀₀₀-DSer-beta-ALAL-Dox and PEG₂₀₀₀-(DSer)₄-beta-ALAL-Dox on MCF-7/6 cells. The survival of cells was estimated by a cell viability test (WST-1, Roche Molecular Diagnostic, Mannheim, Germany). Graphs 2 A, B, C, D and E show the survival of MCF 7/6 cells (in % of the control) respectively based on the logarithm of the doxorubicin concentration (A), beta-ALAL-Dox (B), PEG₂₀₀₀-beta-ALAL-Dox (C), PEG₂₀₀₀-DSer-beta-ALAL-Dox (D) and PEG₂₀₀₀-(DSer)₄-beta-ALAL-Dox (E).

FIG. 3 graphically represents the variations in the mean body weights of xenografted mice carrying LS-174T tumors. The results are expressed in percentage of the weights that are measured at the beginning of the treatment: (●) NaCl, (▪) doxorubicin 6.69 μmol/kg, (▴) doxorubicin 8.6 μmol/kg, (o) Suc-beta-ALAL-Dox 45 μmol/kg, (+) Suc-beta-ALAL-Dox 50 μmol/kg, (x) PEG₂₀₀₀-(DSer)₄-ALAL-Dox 1×45; 3×25 μmol/kg, (*) PEG₂₀₀₀-(DSer)₄-ALAL-Dox 1×50; 3×35 μmol/kg.

FIG. 4 graphically shows the variations in the median relative tumor volumes (RTV) by percentage as of the beginning of daily treatment, of groups of athymic mice carrying LS-147T human colon cancer tumors that are treated relative to the control (NaCl): (●) NaCl, (▪) doxorubicin 6.69 μmol/kg, (▴) doxorubicin 8.6 μmol/kg, (o) Suc-beta-ALAL-Dox 45 μmol/kg, (+) Suc-beta-ALAL-Dox 50 μmol/kg, (x) PEG₂₀₀₀-(DSer)₄-ALAL-Dox 1×45; 3×25 μmol/kg, (*) PEG₂₀₀₀-(DSer)₄-ALAL-Dox 1×50; 3×35 μmol/kg.

FIG. 5 shows the inhibition of the first and second growth phases of LS-174T tumors by doxorubicin (Dox), PEG₂₀₀₀-(DSer)₄-ALAL-Dox and Suc-beta-ALAL-Dox with respective doses of 6.69 μmol/kg, 1×50+3×35 μmol/kg, and 50 μmol/kg, in comparison with the control solution of NaCl 0.9% (w/v). All the mice received 4 i.v. injections on days 0, 7, 14, and 21. The minimum T/C ratios of medians (T/C min.) of relative tumor volumes (RTV) are provided as a maximum effectiveness parameter. The time differences for doubling the medians of the RTV of the groups that are treated in comparison to the control group (T-C) as well as the SGD (specific growth delays) are calculated from the linear regression of the growth phase to determine the degree of activity according to the criteria established by the EORTC. The linear ratio of the slopes of regressions of the variations of the medians of the RTV of treated groups relative to the control group (T/C slope), expressed in percent, are provided by way of the growth rate comparison parameter.

FIG. 6 shows an in vitro stability test of beta-ALAL-Dox (A), PEG₂₀₀₀-beta-ALAL-Dox (B) and PEG₂₀₀₀-(DSer)₄-beta-ALAL-Dox (C) in the serum-containing media. Based on time, the results represent the concentrations of conjugates determined by HPLC.

FIG. 7 shows an in vitro stability test of the compounds ALAL-Dox (A), beta-ALAL-Dox (B), PEG₂₀₀₀-beta-ALAL-Dox (C) and PEG₂₀₀₀-(DSer)₄-beta-ALAL-Dox (D) in whole human blood. The results represent the variations based on time of the concentrations of conjugates and possible metabolites formed, determined by HPLC.

FIG. 8 graphically shows the variations of the mean body weights of athymic mice carrying HCT-116 human colon cancer tumors. The results are expressed in percentage of weight measured at the beginning of the treatment: (●) NaCl, (+) Suc-beta-ALAL-Dox 30 μmol/kg; (▴) PEG₂₀₀₀-(DSer)₄-ALAL-Dox 53 μmol/kg; (*) PEG₂₀₀₀-(DSer)₄-ALAL-Dox 110 μmol/kg.

FIG. 9 shows the inhibition of the HCT-116 tumor growth by the variations of the ratio between the median RTV of treated groups (T) and that of control group (C) that is expressed in percentage (T/C(%)). Treatments: (●) NaCl, (+) Suc-beta-ALAL-Dox 30 μmol/kg; (▴) PEG₂₀₀₀-(DSer)₄-ALAL-Dox 53 μmol/kg; (*) PEG₂₀₀₀-(DSer)₄-ALAL-Dox 110 μmol/kg.

FIG. 10 graphically shows the variations of the survival of xenografted mice carrying HCT-116 tumors (in %). Treatments: PEG₂₀₀₀-(DSer)₄-ALAL-Dox:, 300 μmol/kg (--▴--), 400 μmol/kg (-▴-); PEG₂₀₀₀-ALAL-Dox: 400 μmol/kg (——▪——).

FIG. 11 shows the inhibition of the HCT-116 tumor growth by the variations of the ratio between the median of the RTV of treated groups (T) and that of control group (C) that is expressed by percentage (T/C(%)). NaCl (-●-), PEG₂₀₀₀-(DSer)₄-ALAL-Dox 200 μmol/kg (......▴......), PEG₂₀₀₀-ALAL-Dox 200 μmol/kg (......▪......).

FIG. 12 graphically shows the variations of the survival of xenografted mice carrying B16-BL6 melanoma tumors (%). Treatments: Control PBS (●); (PEG₅₀₀₀-ALAL)ₙ-TNFα(▪); (PEG₅₀₀₀-(DSer)₄-ALAL)ₙ-TNFα (▴); (PEG₅₀₀₀)ₙ-TNFα (♦).

FIG. 13 graphically shows the variations of the mean body weights of xenografted mice carrying B16-BL6 melanoma tumors. The results are expressed in percentage of weight measured at the beginning of the treatment. Control PBS (●); (PEG₅₀₀₀-ALAL)ₙ-TNFα˜ (▪); (PEG₅₀₀₀-(DSer)₄-ALAL)ₙ-TNFα (▴); (PEG₅₀₀₀)ₙ-TNFα (♦).

FIG. 14 shows the inhibition of B16-BL6 tumor growth by the variations of the ratio between the median RTV of treated groups (T) and that of control group (C) that is expressed by percentage (T/C(%)). Control PBS (●); (PEG₅₀₀₀-ALAL)ₙ-TNFα (▪); (PEG₅₀₀₀-(DSer)₄-ALALₙ-TNFα (▴); (PEG₅₀₀₀)ₙ-TNFα (♦).

FIG. 15 graphically shows the distribution of Doxorubicin □ and Leu-doxorubicin ▪ in the tumor of LS-174T tumor bearing nude mice, A: one hour and B: 3 hours following IV injection of the PEG_{30,000}(DSer)₈-ALAL-Dox or PEG_{30,000}-ALAL-Dox at 80 μmol/kg

FIG. 16 shows in vitro reactivation of ester PEG_{20,000}-(DSer)₈-ALAL-Dox and ester PEG_{20,000}-ALAL-Dox conjugates following incubation in the presence of LS-174T tumor cells conditioned medium. Graph is expressed as the sum of (Dox+Leu-Dox+Ala-Leu-Dox) concentrations cleaved from an equimolar concentration of the initial compounds as function of the incubation time. Ester PEG_{20,000}-(DSer)₈-ALAL-Dox ●, ester PEG_{20,000}-ALAL-Dox X.

### DETAILED DESCRIPTION OF PREFERRED EMBODIMENTS

This invention is directed to a new prodrug structure to eliminate this steric hindrance phenomenon (i.e. the steric hindrance phenomenon stabilizing or masking group of high molecular weight) and to make possible or to facilitate the cleavage of the oligopeptide when the masking and/or stabilizing group is large, while keeping a high specificity of action, a low toxicity, and a stability in the blood and/or serum, preferably in a mammal. In a preferred embodiment, the mammal is a human.

This object is attained by inserting a “molecular arm” or a “molecular spacer” between the masking and/or stabilizing group (for example the PEG), and the peptide sequence that can be cleaved by an enzyme “specific” of this sequence.

The molecular spacers according to the invention, also referred to below as “spacers,” were selected by taking into consideration hydrophilic properties of the units that constitute the spacer.

This invention therefore first has as its object a compound of formula (A)ₚ-(E-B)ₙ-(I)ₘ:

in which:
I is an active substance of interest against target cells,
A is a group that increases the half-life time of B-I in the blood circulation,
E-B is a group connecting A and I, where:
B is a structure that can be cleaved selectively by an enzyme that is present only or preferably close to or at said target cells,
E is a hydrophilic spacer group, stable in the circulatory structure, which separates A from B so as to make possible or to facilitate the cleavage of B close to or at said target cells and thus to make possible or facilitate the release of I or the release of I with a radical or fragment of B,
n is an integer between 1 and either the total number of reactive functions of I on which connecting groups E-B can be coupled, or the total number of reactive functions of A on which connecting groups E-B can be coupled,
m is an integer between 1 and the total number of reactive functions of A on which connecting groups E-B can be coupled, or the total number of reactive functions of B on which I can be coupled
p is an integer between 1 and the total number of reactive functions of I, on which connecting groups E-B can be coupled, or the total number of reactive functions of E on which A can be coupled

with, optionally, when p=1, n=m, and when m=1, n=p.

According to a first preferred embodiment of this invention, p, n and m are equal to 1. The compound is then shown by the following formula A-E-B-I.

According to a second preferred embodiment of this invention, m is equal to 1, and n and p are identical and more than 1. The compound is then shown by the following formula (A-E-B)_{t>1}-I, where t represents an integer between 2 and the total number of reactive functions of I on which group A-E-B- can be coupled. Advantageously, I can be represented by a polypeptide, such as a TNF-alpha cytokine molecule on which several A-E-B- groups are covalently bonded.

According to a third preferred embodiment of this invention, p is equal to 1, and n and m are identical and more than 1. The compound is then shown by the following formula A-(E-B-I)_{k>1}, where k represents an integer between 2 and the total number of reactive functions of A on which group -E-B-I can be coupled. Advantageously, A can be represented by a polymer, such as a branched PEG molecule, on which are branched several groups -E-B-I.

According to a fourth preferred embodiment of this invention, p and m are more than 1, and n can be different from p and m. By way of example, if p=2, n=3 and m=2, the compound can be represented by the following formula:
(I)-(B-E)-(A)-(E-B)-(I)-(B-E)-(A)

The active substance of interest (I) can be attached either directly to one or more structures B by a covalent bond, or indirectly via a “connecting arm”. The term “connecting group” means a divalent organic radical selected from substituted or unsubstituted alkyl, substituted or unsubstituted heteroalkyl and substituted or unsubstituted aryl. By way of example, when structure B is a sequence of amino acids and when it is attached directly to substance of interest I, the covalent bond can then be produced at the N-terminal or C-terminal end of the amino acid sequence according to its orientation, or at any other site of the oligopeptide (for example at the lateral chain of one of the amino acids). Furthermore, when the bond between B and I is indirect, the connecting arm can have several functions such as to facilitate the cleavage between B and I, to provide a suitable chemical bonding means between B and I, to improve the synthesis process of the compound, to improve the physical properties of the substance of interest (I), or to provide an additional mechanism of the intracellular or extracellular release of substance of interest (I). This indirect bond can be carried out by any chemical, biochemical, enzymatic or genetic coupling process that is known to one skilled in the art. By way of example of such connecting arms, it is possible to cite a homofunctional or heterofunctional bridging reagent such as succinimidyl 4-(N-maleimidomethyl)cyclohexane-1-carboxylate; bi- or multifunctional agents that contain alkyl, aryl, aralkyl or peptide groups; esters, aldehydes or alkyl, aryl or arylalkyl acids; anhydride groups, sulfhydril groups or carboxyl groups, such as the derivatives of maleymil benzoic acid, maleymil propionic acid and succinimidyl derivatives; cyanogen bromide or chloride derivative groups; carbonyldiimidazole, thiocarbonyldiimidazole of succinimide esters or sulfonic halides; phosgene, thiophosgene; or self-rearrangeable (or “self-immolative”) spacers (Schmidt et al., 2001).

Spacer (E) according to this invention links group A to structure B. It is preferably hydrophilic and stable in the circulatory structure (e.g. the blood circulation or bloodstream).

A spacer is “stable in the circulatory structure” when less than about 20%, preferably less than about 10%, preferably even less than about 2%, of the spacer is degraded or cleaved (in particular by enzymes) in the circulating blood or during its preservation in human blood at about 37° C., for more than about 2 hours. Advantageously, by separating group A from structure B and by its preferably hydrophilic nature, the spacer makes possible or facilitates the cleavage of structure B close to or at target cells and thus makes possible or facilitates the release of I or the release of I with a B radical or fragment. The spacer can exhibit a length size that can be on the order of the equivalent of about 1 to about 100 amino acids, preferably of about 1 to 20.

According to another aspect of this invention, the size of the spacer can vary based on the molecular weight of group A. According to this aspect, the size of the spacer is larger, the higher the molecular weight of A.

The spacer according to this invention consists of, or alternatively comprises, at least one group that is selected from among: the sequences of amino acids; the peptidomimetic agents; the pseudopeptides; the peptoids; the substituted alkyl, aryl or arylalkyl chains; the polyalkyl glycols; the polysaccharides; the polyols; the polycarboxylates; and the poly(hydro)esters. In another embodiment of the invention, the spacer thus can consist of, or alternatively comprise, a combination of at least two of these groups.

According to an advantageous embodiment of the invention, the spacer consists of or comprises about 1 to about 100, preferably about 1 to about 20, and very preferably about 2 to about 10, identical or different amino acids selected from the group that comprises the natural amino acids in conformation D, the genetically uncoded amino acids or the amino acids that cannot be recognized by an enzyme (e.g. the peptidases) that is present in the circulatory structure, such as the beta- or gamma-amino acids or the like. “Natural amino acids in conformation D” are defined as the amino acids that are normally coded by the genetic code but that instead of being naturally in conformation L are in conformation D. Generally, the genetically uncoded amino acids can be prepared by synthesis or can be derived from a natural source.

Preferred among the natural amino acids in conformation D are the hydrophilic amino acids that are selected from among: D-glutamine, D-asparagine, D-aspartic acid, D-glutamic acid, D-lysine, D-arginine, and D-histidine. Particularly preferred amino acids in conformation D are D-serine and D-threonine.

According to a preferred method of this invention, the spacer comprises, or alternatively consists of, a sequence of identical amino acids that are selected from among: (D-serine)ₓ or (D-threonine)ₓ, where x is an integer between about 1 and about 20, preferably between about 2 and about 10, and very preferably between about 2 and about 6.

Preferred spacers comprise, or alternatively consist of, the following:

(D-serine)-(D-serine)-(D-serine)-(D-serine), equally noted Dseyl-Dseyl-Dseyl-Dseyl, or

(D-threonine)-(D-threonine)-(D-threonine)-(D-threonine), equally noted DThreonyl-DThreonyl-DThreonyl-DThreonyl, or

(D-serine)₈, equally noted (DSer)₈ or

(D-serine)₁₂, equally noted (DSer)₁₂.

The referenced amino acids are also represented in this invention either in three-letter code or in one-letter code, and it is submitted that three-letter and one letter amino acid code are well known to one skilled in the art.

Group A is a group that increases in vivo the half-life time of B-I in the circulatory structure. This result is attained in particular when A reduces the renal elimination of substance of interest I or of compound B-I, whereby this elimination is based on the ultrafiltration through the kidneys of the compounds based on size. Thus, the larger the compound is, the slower its elimination, whereby the renal elimination is ineffective for compounds having a molecular weight of at least about 50,000 Dalton. This result can also be attained by reducing the degradation by the hepatic metabolism of the compounds according to the invention. In other words, to increase the half-life time is to increase the mean residence time of the compounds in the blood or to reduce the blood or plasmatic clearance.

“Circulatory structure” is defined as body fluids, more particularly blood. In a specific embodiment the “circulatory structure” is those of a mammal, including tissues of the circulatory system.

Structures encompassed by Group A are preferably hydrophilic or amphipathic.

Group A is preferably stable in the circulatory structure (i.e., when less than about 20%, preferably less than about 2%, of the compound of formula (A)ₚ-(E-B)ₙ-(I)ₘ is degraded or cleaved in the circulating blood (in particular by enzymes), during its preservation in human blood (at about 37° C. for more than about 2 hours), is non-toxic for healthy cells, non-immunogenic, non-coagulating (i.e. prevents pro-coagulating properties of the moiety B-I) or masking (i.e., preventing substance of interest (I) from acting on the surface of a cell until the latter has been released from the prodrug).

Advantageously, group A can also have one or more of the following properties: prevent the non-specific cleavage and/or the degradation of connecting group E-B; inhibit the biological effects of the substance of interest until the substance of interest has been released from the prodrug; increase the stability of the compound in the circulatory structure; increase the solubility (or increase the solubilization) in water, blood and/or the serum of the compound of formula (A)ₚ-(E-B)ₙ-(I)ₘ; and enhance the targeting (or targeting-enhancing) properties of the compound of formula (A)ₚ-(E-B)ₙ-(I)ₘ toward the target cells.

By “Properties of targeting the compound of formula (A)ₚ-(E-B)ₙ-(I)ₘ toward the target cells” is intended as group A making it possible for the compound of formula (A)ₚ-(E-B)ₙ-(I)ₘ to accumulate close to or at the target cells. Group A will then be called “biospecific,” i.e., able to develop specific biological interactions and consequently to be recognized by biological entities of the living system. In particular, it can be grafted to the surface of group A peptide sequences such as antibodies, antigens or groups of amino acids such as arginine-glycine-aspartic acid (RGD), making it possible to selectively increase the adhesion of the compound according to the invention to the surface of certain cell types. Group A can also consist of a biospecific copolymer for functionalization of pre-existing macromolecular chains by suitable chemical groups for the purpose of being recognized as a cellular marker or cellular recognition sequence, or else by copolymerization of functionalized monomers.

In a preferred embodiment, group A is selected from among the group of: the polypeptides (such as polyglutamate, polyaspartate), the immunoglobulins, albumin, polysaccharides, polymers or copolymers.

The term “immunoglobulin” according to the present invention, refers to immunoglobulin molecules and immunologically active portions of immunoglobulin molecules, i.e., molecules that contain an antigen binding site that immunospecifically binds an activated RAS protein. The immunoglobulin molecules of the invention can be of any type (e.g., IgG, IgE, IgM, IgD, IgA and IgY), class (e.g. IgG1, IgG2, IgG3, IgG4, IgA1 and IgA2) or subclass of immunoglobulin molecule. The term also includes, but is not limited to, Fab, Fab′ and F(ab′)2, Fd, single-chain Fvs (scFv), single-chain antibodies, disulfide-linked Fvs (sdFv) or nanobodies (i.e. antibody-derived therapeutic proteins that contain structural and functional properties of naturally-occurring heavy-chain antibodies; such as those described in PCT patent application published under No WO 04/041862, WO 04/041863, WO 04/041865) and fragments comprising either a VL or VH domain.

The polymers can comprise, or alternatively consist of, polyvinyl pyrrolidone, the copolymer pyran, polyhydroxypropyl-methacrylamide-phenol, polyhydroxy-ethyl-aspanamide-phenol, poly(ethylene oxide)-polylysine substituted by palmitoyl residues, poly(lactic acid), poly(epsilon-caprolactone), poly(hydroxybutyric acid), polyorthoesters, polyacetals, polydihydropyrans, polycyanoacrylates and the sequenced hydrogel copolymers that are crosslinked or amphipathic.

Group A is preferably selected from among the following group of: a polyalkylene glycol, polyalkylene oxide, polyalkylene imine, and vinyl chloride copolymers. By way of a non-limiting example, when group A is a vinyl chloride copolymer, the presence of sulfonate groups or sulfonate and carboxylate is necessary so that the polymer does not develop a coagulating activity.

Group A is preferably also selected from among or is a combination of the following group of: a polyethylene oxide, a polyethylene imine, sodium styrene sulfonate (NaSS), sodium maleate and butyl maleate (MMBE), hydroxypropyl methacrylate or N-(2-hydroxypropyl)methacrylamide (HPMA), methyl methacrylate (MMA), poly-[N-(2-hydroxyethyl)-L-glutamine] (PHEG), poly-[N-(hydroxyethyl)-DL-aspartamide] (PHEA), or polylactic acid (PLA).

In a preferred embodiment the group A comprises polyethylene glycol (PEG) and polylactic acid moieties.

In a preferred embodiment, the size of the group A can be between about 200 and about 50,000 Da, preferably between about 350 and about 20,000 Da and preferably also between about 1,000 and about 10,000 Da.

In a more preferred embodiment group A is a polyethylene glycol (PEG). PEG is made up of repeating units of —(CH₂CH₂O)ₙ— wherein n can be between about 40 and 1200. The size of the PEG can be between about 200 Da and about 50,000 Da, and specifically can be about 500 Da, 700 Da, 1,000 Da, 2,000 Da, 3,000 Da, 4,000 Da, 5,000 Da, 8,000 Da, 10,000 Da, 15,000 Da, 20,000 Da, 30,000 Da, 40,000 Da or 50,000 Da. In one embodiment, the size of the PEG is between about 20,000 and 50,000 Daltons. The presence of these PEG makes possible an improvement of pharmacokinetic properties (Duncan et al., 1994), and pharmacodynamic properties and therefore a reduction of the renal elimination of the compounds according to the invention. Furthermore, another known advantage of the prior art is that PEG makes possible a preferred accumulation in the tumors. Actually, the PEGs of a molecular weight of 10⁴ Da or more show a more significant accumulation in the tumors than in the normal tissues (Greenwald et al., 2003; Seymour et al, 1995).

In a specific embodiment, the term “polyethylene glycol” comprises any PEG derivative such as an ester derivative of polyethylene glycol PEG (i.e. an esterified PEG). A preferred PEG ester derivative is a PEG esterified to the succinimidyl succinate (see example 14).

Group A can also be an agent with therapeutic activity or an agent with diagnostic activity.

Advantageously, a group A that has properties of an agent with diagnostic activity further comprises paramagnetic properties, i.e., in its electronic layers, it can have single electrons such as gadolinum, manganese or iron that reinforce in particular the contrast in magnetic resonance imagery (MRI). By way of a non-limiting example, it is possible to cite the polystyrene sulfonate to which the iron is attached.

The invention also encompasses compounds that use one or more targeting substances that can vector the compound of formula (A)ₚ-(E-B)ₙ-(I)ₘ toward the target cells. In one embodiment of the invention, the targeting substance comprises, or alternatively consists of, one or more of antibodies, antigens, or liposomes.

The targeting substance or substances is (are) preferably coupled with the compound of formula (A)ₚ-(E-B)ₙ-(I)ₘ at one or more group(s) A.

According to this invention, structure B can be cleaved selectively by an enzyme that is present only or preferably in the environment of the target cells.

“Target cells” are defined more particularly as the cells that are involved in pathology, that exhibit a therapeutic or diagnostic advantage, or that are preferred targets for therapeutic or diagnostic activity. These target cells are preferably one or more of the cells comprising, or alternatively consisting of, the cells of the following group: primary or secondary tumor cells (the metastases), stromal cells of primary or secondary tumors, neoangiogenic endothelial cells of tumors or tumor metastases, macrophages, monocytes, polymorphonuclear leukocytes and lymphocytes or polynuclear agents infiltrating the tumors and the tumor metastases.

“Selectively cleavable” is defined as cleavage dictated by the sequence to be cleaved. In other words, the sequence to be cleaved is preferably recognized by an enzyme that is present in the environment of the target cells, and it is degraded slightly or not at all in the circulatory structure or close to non-target cells. The expression “in the environment of the target cells” means that the enzyme is present either by itself or preferably close to or at the target cells. It should be noted that even if the cleavage is not carried out only close to or at the target cells, the fact that the cleavage is carried out preferably (or in large part or in the majority of the cases) close to or at the target cells makes this cleavage selective. In other words, the cleavage is called selective when the enzyme is in a larger concentration close to or at the target cells relative to the remainder of the organism.

In a specific embodiment, the term “enzyme” is defined as a hydrolase. The enzyme comprises, or alternatively consists of, one or more enzymes selected from the group of peptidases, endopeptidases, lysosomal enzymes, lipases and glycosidases.

According to a preferred embodiment of this invention, the enzyme is a peptidase of the tumor cells, stromal cells of the tumors, neoangiogenic endothelial cells, macrophages or monocytes. “Specific enzyme” is defined as a membrane enzyme or an enzyme that is secreted only or preferably by the target cells in the extracellular medium of these target cells. In one embodiment of the invention, when the enzyme is specific to tumor cells, the latter can be selected from the group that comprises, or alternatively consists of, neprilysine (CD10), thimet oligopeptidase (TOP), prostate specific antigen (PSA), plasmine, legumaine, collagenases, urokinase, cathepsins, and the matrix metallopeptidases.

The selection of structure B is based on, of course, the enzyme that is present in the environment of the target cells. Thus, if the enzyme is a peptidase, structure B will comprise, or alternatively consist of, a sequence of amino acids (or oligopeptide) that can be cleaved by this peptidase; if the enzyme is a glycosidase (for example a saccharase), then structure B will comprise, or alternatively consist of, an oligosaccharide that can be cleaved by this glycosidase; if the enzyme is a lipase, then structure B will comprise, or alternatively consist of, a lipid chain that can be cleaved by this lipase, and so on.

The object of the invention is preferably a structure B that comprises, or alternatively consists of, an oligopeptide that can be cleaved selectively by an enzyme that is present in the environment of tumor cells. The oligopeptide preferably comprises, or alternatively consists of, between about 2 and about 10 amino acids, and preferably also about 3 to about 7 amino acids. In a preferred embodiment of the invention, the oligopeptide comprises, or alternatively consists of, one or more of the following sequences (preferably in conformation L):

(SEQ ID No. 1)

Ala-Phe-Lys,

(SEQ ID No. 2)

Ala-Leu-Ala-Leu
or

(SEQ ID No. 3)

beta-Ala-Leu-Ala-Leu, Ala-Leu-Lys-Leu-Leu,

(SEQ ID No. 4)

Ala-Tyr-Gly-Gly-Phe-Leu,

(SEQ ID No. 5)

His-Ser-Ser-Lys-Leu-Gln-Leu,

(SEQ ID No. 6)

Gly-Pro-Leu-Gly-Ile-Ala-Gly-Gln,

(SEQ ID No. 7)

Cys-Asp-Cys-Arg-Gly-Asp-Cys-Phe-Cys
and

(SEQ ID No 8)

Ala-Leu-Lys-Leu-Leu.

One skilled in the art, however, knows other sequences of amino acids that can be cleaved selectively by a specific enzyme of tumor cells, such as those that are described in U.S. Pat. No. 5,962,216, No. 6,342,480, No. 6,844,318, No. 6,897,034 or U.S. patent applications Ser. No. 10/879,442, Ser. No. 10/296,954, Ser. No. 10/311,411, Ser. No. 10/879,442, Ser. No. 10/311,519, Ser. No. 10/333,619 or Ser. No. 10/641,667.

The enzymes according to the invention are able to selectively cleave structure B so as to make possible the release of I or the release of I with a radical or fragment of B. The expression “release of I with a radical or fragment of B” is explained by the following example. If structure B is an amino acid sequence whose sequence is Ala-Leu-Ala-Leu, the substance of interest is doxorubicin (whereby B-I is Ala-Leu-Ala-Leu-Dox) and the enzyme is CD10, then this enzyme will cleave the sequence of amino acids between Ala-Leu-Ala and Leu, thus releasing a Leu-doxorubicin product. This product is therefore defined as “I with a radical or fragment of B.”

“Extra-blood reactivation” or “reactivation in the extra-blood compartment” is defined as the cleavage of peptide bond B of the prodrug of structure (A)p-(E-B)n-(I)m by specific endopeptidases that are present in any organ or tissue (healthy or tumor, for example) other than the blood and preferably at target cells. The cleavage of structure B (for example a peptide) results in the release of an active form of the substance of interest (I) (for example a therapeutic agent).

“Active substance of interest against target cells” (I) is defined as a substance whose action site is located or whose effect will be exerted on the surface or inside target cells. By way of example, such a substance of interest comprises, or alternatively consists of, an agent selected from the following group: a chemical agent, a polypeptide, a protein, a nucleic acid (DNA, sense or antisense RNA, single or double strand, complementary DNA, interfering RNA, and the like), an antibiotic, and a virus or a marker, optionally coupled with a vector substance (for example an antibody).

Said substance of interest (I) is preferably an agent with therapeutic activity, and more preferably an agent with anti-tumor, anti-angiogenic or anti-inflammatory therapeutic activity. The agent can have a target (for example a receptor) or extracellular or intracellular action site. It can also comprise a penetrating peptide sequence such as a sequence that is described in U.S. patent application Ser. No. 10/231,889. By way of example, the substance of interest (I) comprises, or alternatively consists of, a substance of interest (I) selected from the following group of agents with anti-tumor therapeutic activity: vinca alkaloids such as vincristine, vinblastine, vindesine, vinorelbine; taxanes or taxoids such as paclitaxel, docetaxel, 10-deacetyltaxol, 7-epi-taxol, baccatin III, le xylosyltaxol; alkylating agents such as ifosfamide, melphalan, chloroaminophene, procarbazine, chlorambucil, thiophosphoramide, busulfan, dacarbazine (DTIC), mitomycins including mitomycin C, nitroso-ureas and derivatives thereof (for example, estramustine, BCNU, CCNU, fotemustine); platinum derivatives such as cisplatin and the like (for example, carboplatin, oxaliplatin); antimetabolites such as methotrexate, aminopterin, 5-fluorouracil, 6-mercaptopurine, raltitrexed, cytosine arabinoside (or cytarabine), adenosine arabinoside, gemcitabine, cladribine, pentostatin, fludarabine phosphate, hydroxyureas; inhibitors of topoisomerase I or II such as the camptothecin derivatives (for example, irinotecan and topotecan or 9-dimethylaminomethyl-hydroxy-camptothecin hydrochloride), epipodophyllotoxins (for example etoposide, teniposide), amsacrine; mitoxantrone; L-canavanine; antibiotic agents such as anthracyclins and, for example, adriamycin or doxorubicin, THP-adriamycin, daunorubicin, idarubicin, rubidazone, pirarubicin, zorubicin and aclarubicin, the analogs of anthracyclins and, for example, epiadriamycin (4′epi-adriamycin or epirubicin) and mitoxantrone, bleomycins, actinomycins including actinomycin D, streptozotocin, calicheamicin, duocarmycins, combretastatin; L-asparaginase; hormones; pure inhibitors of aromatase; androgens, analog-antagonists of LH-RH; cytokines such as interferon alpha (IFN-alpha), interferon gamma (IFN-gamma), interleukin I (IL-1), IL-2, IL-4, IL-6, IL-10, IL-12, IL-15, the tumor necrosis factor-alpha (TNF-alpha), the IGF-1 antagonists (insulin-like growth factor); the proteasome inhibitors; the farnesyl-transferase inhibitors (FTI); the epothilones; the maytansinoids; discodermolide; fostriecin; BH3 peptides; p53 peptides; caspases; granzyme B; ribozymes; monoclonal antibodies such as rituximab, tastuzumab; the inhibitors of tyrosine kinases such as STI 571 (imatinib mesylate); andostatins; proteins, peptides, and anti-inflammatory cytokines.

“Markers” are defined as enzymes, antibodies, fluorescent or phosphorescent chemical molecules, and molecules that can be used in scintigraphy. Examples of markers include, but are not limited to, coumarin, 7-amido-trifluoromethyl coumarin, paranitroanilide, 8-naphthylamide and 4-methoxy naphthylamide, fluorosceine, biotin, rhodamine, tetramethylrhodamine, GFP (green fluorescent protein), the agents that are used in scintigraphy as radioactive isotopes, and the derivatives of these compounds.

In another embodiment of the invention, the invention encompasses pharmaceutically acceptable basic or acidic addition salts, hydrates, solvates, precursors, metabolites or stereoisomers of said compounds according to this invention.

The expression “pharmaceutically acceptable salts” refers to non-toxic salts of the compounds according to the invention that can generally be prepared by reacting a free base of the compound according to the invention with a suitable organic or inorganic acid. These salts preserve the biological effectiveness and the properties of free bases. In one embodiment of the invention, salts comprise, or alternatively consist of, one or more of the salts selected from the following group: water-soluble salts and water-insoluble salts, such as acetates, ansonates (4,4-diaminostilbenes-2,2′-disulfonates), benzenesulfonates, benzonates, bicarbonates, bisulfates, bitartrates, borates, bromides, buryrates, calcium edetates, camsylates, carbonates, chlorides, citrates, clavulariates, dichlorohydrates, edetates, edisylates, estolates, esylates, fumarates, gluceptates, gluconates, glutamates, glycolylarsanylates, hexafluorophosphates, hexylresorcinates, hydrabamines, bromohydrates, chlorohydrates, hydroxynaphthoates, iodides, isothionates, lactates, lactobionates, laurates, malates, maleates, mandelates, mesylates, methylbromides, methylnitrates, methylsulfates, mucates, napsylates, nitrates, 3-hydroxy-2-naphthoates, oleates, oxalates, palmitates, pamoates (1,1-methylene-bis-2-hydroxy-3-naphthoates, emboates), pantothenates, phosphates/diphosphates, picrates, polyglucuronates (such as polygalacturonates and polyglucuronates), propionates, p-toluenesulfonates, salicylates, stearates, subacetates, succinates, sulfates, sulfosalicylates, suramates, tannates, tartrates, teoclates, tosylates, triethiodides, valerates and N-methylglucamine ammonium salts.

Another embodiment of the invention is also a composition that comprises, or alternatively consists of, as an active ingredient, at least one compound according to this invention. In still another embodiment, the invention contemplates the use of such compositions for the formulation and the preparation of biological, pharmaceutical, cosmetic, agricultural, diagnostic or tracing products.

In a preferred embodiment, the invention encompasses pharmaceutical formulations that comprise, or alternatively consist of, at least one compound according to this invention that can be combined with a pharmaceutically acceptable vehicle, vector, diluent or excipient.

A subject can be treated with a pharmaceutically effective amount of a compound according to the invention. In a preferred embodiment of the invention, the subject is a human subject. The expression “pharmaceutically effective amount” means an amount that can induce the biological or medical response of a tissue, system, animal or human as expected by the research worker or the doctor in attendance.

The compositions defined above can also comprise, or be combined with, at least one other medicinal active ingredient or at least one adjuvant that is well known to one skilled in the art, such as for example vitamin C, anti-oxidizing agents, to be used in conjunction with the compound according to the invention to improve and to extend the treatment.

The compositions of the invention are particularly useful in that they have a very low toxicity or are not toxic.

The pharmaceutical formulations according to the invention are able to be used in vivo for preventive or curative purposes for diseases or disorders. Non-limiting examples of diseases or disorders for which the pharmaceutical formulations according to the invention may be used include viral infections, cancers, metastases, cellular apoptosis disorders, degenerative diseases, tissue ischemia, infectious diseases of a viral, bacterial or fungal nature, inflammation disorders and pathological neo-angiogenesis.

The administration of the compounds according to the invention can be done by any of the administration methods accepted for the therapeutic agents and generally known in the art. These processes include, but are not limited to, the systemic administration, for example by oral, nasal, parenteral or topical administration, for example by transdermal means or else by central administration, for example by an intracranial surgical path, or else by intraocular administration.

The oral administration can be done by means of tablets, capsules, soft capsules (including formulations with delayed release or extended release), pills, powders, granules, elixirs, dyes, suspensions, syrups and emulsions. This form of presentation is more particularly suited for the passage of the intestinal barrier.

The parenteral administration is done generally by subcutaneous, intramuscular or intravenous injection, or by perfusion. The injectable compositions can be prepared in standard forms, either in suspension or liquid solution or in solid form that is suitable for an extemporaneous dissolution in a liquid.

A possibility for parenteral administration uses the installation of a system with slow release or extended release that ensures the maintenance of a constant dose level, for example such as that disclosed in U.S. Pat. No. 3,710,795.

For intranasal administration, it is possible to use suitable intranasal vehicles that are well known to those skilled in the art.

For transdermal administration, it is possible to use transdermal cutaneous patches that are well known to one skilled in the art. A transdermal release system allows for continuous administration. Other preferred topical preparations include, but are not limited to, creams, medicated ointments, lotions, aerosol sprays and gels.

Based on the administration method provided, the compounds can be in solid, semi-solid or liquid form.

For solid compositions, such as tablets, pills, powders or granules in the free state or included in capsules, the active ingredient can be combined with excipients, such as: a) diluents, for example lactose, dextrose, sucrose, mannitol, sorbitol, cellulose and/or glycine; b) lubricants, for example silica, talc, stearic acid, its magnesium or calcium salt and/or polyethylene glycol; c) binders, for example magnesium silicate and aluminum silicate, starch paste, gelatin, tragacanth gum, methyl cellulose, carboxymethyl cellulose that contains soda and/or polyvinyl pyrrolidone; if necessary, d) disintegrating agents, for example starch, agar, alginic acid or its sodium salt, or effervescent mixtures; and/or e) absorbents, coloring agents, aromatizing agents and sweetening agents.

For semi-solid compositions, such as suppositories, the excipient can be, for example, a fatty emulsion or suspension or can be based on polyalkylene glycol, such as polypropylene glycol.

The liquid compositions, in particular those intended for injection or to be included in a soft capsule, can be prepared by, for example, dissolution, dispersion, etc., of the compound according to the invention in a pharmaceutically pure solvent such as, for example, water, a saline solution of sodium chloride (NaCl), the physiological serum, aqueous dextrose, glycerol, ethanol, an oil and the like.

The compounds according to the invention can also be administered in the form of systems for release of the liposome or lipoplex type, such as in the form of small unilamellar vesicles, large unilamellar vesicles and multilamellar vesicles. The liposomes can be formed from a variety of phospholipids, containing cholesterol, stearylamine or phosphatidylcholines. In one embodiment, a liquid component film can be hydrated with an aqueous solution of the medication to form a lipid layer that encapsulates the medication, such as described in U.S. Pat. No. 5,262,564.

The compositions according to the invention can be sterilized and/or can contain one or more of: non-toxic adjuvants and auxiliary substances such as agents for preservation, stabilization, wetting or emulsification; agents that promote dissolution; and salts to regulate osmotic pressure and/or buffers. In addition, they can also contain other substances that offer a therapeutic advantage. The compositions are prepared, respectively, by standard processes of mixing, granulation or coating well known to those skilled in the art.

The dosage for the administration of compounds according to the invention is selected according to a variety of factors including the type, strain, age, weight, sex and medical condition of the subject; the severity of the condition to be treated; the method of administration; the condition of the renal and hepatic functions of the subject and the nature of the particular compound or salt that is used. A normally experienced doctor or veterinarian will easily determine and prescribe the effective amount of the desired compound to prevent, disrupt or stop the progress of the medical condition that is to be treated.

Any of the pharmaceutical compositions above can contain from about 0.1 to about 99%, preferably from about 1 to about 70%, of active ingredient.

By way of examples, the oral dosages of the compounds according to the invention, when they are used for the indicated results, will be encompassed between about 0.05 and about 5000 mg/day, preferably between about 5 and about 1000 mg/day when given orally, and preferably provided in the form of tablets that contain about 0.5, about 1.0, about 2.5, about 5.0, about 10.0, about 15.0, about 25.0, about 50.0, about 100.0, about 250.0, about 500.0 and about 1000.0 mg of active ingredient. When given parenterally, the effective levels of the compounds according to the invention will be in the range of from about 0.002 mg to about 500 mg per kg of body weight and per day.

The compounds according to the invention can be administered in the form of single daily doses, or the total daily dosage can be administered in two, three, four or more doses per day.

In a particular embodiment of the invention is provided a diagnostic agent, for use in vitro, comprising or alternatively consisting of at least one compound according to this invention. The compound according to the embodiment will then have a marker as a substance of interest (I). Such a diagnostic agent can also be used in vivo.

This invention also contemplates in another embodiment a diagnostic kit that comprises said diagnostic agnt. More particularly, the diagnostic kit comprises, in one or more containers, a predetermined amount of a composition according to the invention.

Other advantages and characteristics of the invention will emerge from the following examples, given by way of illustration, and in which reference will be made to the accompanying drawings.

### EXAMPLES

### Example 1A

### Material and Methods

1.1) Cell Lines

MCF 7/6 cells: a variant of the MCF-7 cell line (Michigan Cancer Foundation Engel et al., 1978) that was obtained in 1970 from a pleural effusion in a patient suffering from an adenocarcinoma of the chest (Soule et al, 1973). These cells were obtained from the laboratory of Professor Mareel in Ghent (Laboratoire de Cancérologie Expérimentale [Experimental Cancerology Laboratory], Hôpital universitaire de Gand [Ghent University Hospital], Belgium).

LNCaP cells: isolated in 1977 by Horoszewic et al., from a biopsy at the level of supraclavicular lymphatic nodules of a patient suffering from a metastatic carcinoma of the prostate. This line was obtained from the ATCC (American Type Culture Collection. Manassas, Va., USA).

LS-174T cell line: variant of the LS180 line, obtained from a female suffering from a colon adenocarcinoma. These cells form very quickly from tumors after an inoculation in athymic mice. These cells were obtained from the ECACC (European Collection of Cells Cultures. UK).

HCT-116 cells: cell line established from a primary culture of carcinoma cells of the human colon. These cells form tumors after subcutaneous injection in athymic mice. These cells were obtained from the ATCC (American Type Culture Collection, Manassas, Va., USA).

1.2) Anti-Cancer Agents for Chemotherapy Studies

Doxorubicin was provided by Meiji Seika Kaisha Ltd. (Tokyo, Japan); succinyl-beta-Ala-Leu-Ala-Leu-doxorubicin (Suc-beta-Ala-Leu-Ala-Leu-Dox) (Fernandez et al., 2001), PEG₂₀₀₀-Ala-Leu-Ala-Leu-Dox and PEG₂₀₀₀-(DSer)₄-Ala-Leu-Ala-Leu-Dox were synthesized.

1.3) The Animals

NMRI or SWISS female mice, nude/nude (5 weeks after delivery by the JANVIER breeding farms, France) were used.

All handling of the animals was done in accordance with the recommendations of the UKCCCR (United Kingdom Coordinating Committee on Cancer Research; Workman et al., 1998) and the FELASA (Federation of European Laboratory Animal Science Associations; Nicklas et al., 2002; Rehbinder et al., 2000; Rehbinder et al., 1996) on the use and well-being of animals in experimental chemotherapy studies.

### Example 1B

### Synthesis of Prodrug PEGylated Derivatives of Doxorubicin

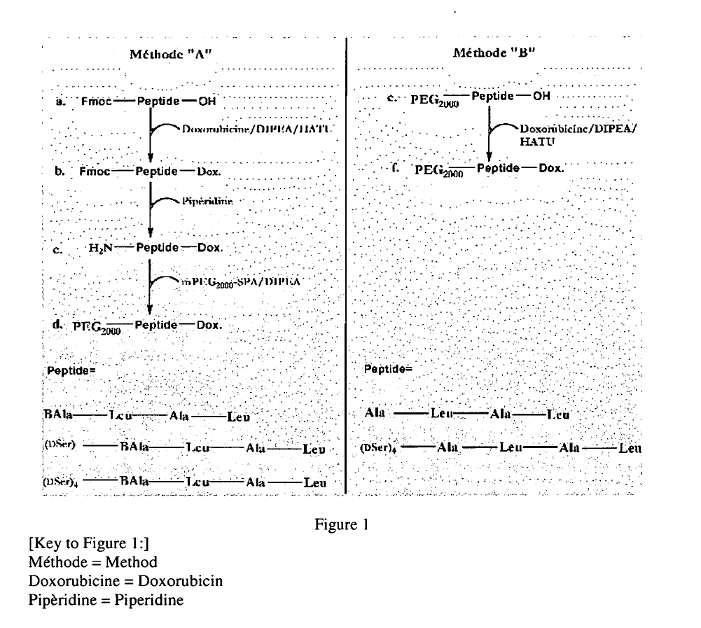

The synthesis of prodrug PEGylated derivatives of doxorubicin was carried out by using two different methods “A” and “B” (FIG. 1).

The synthesized prodrug PEGylated derivatives of doxorubicin according to one or the other of the two methods are as follows:

| | |
|---|---|
| PEG₂₀₀₀-(DSer)4-beta-Ala-Leu-Ala-Leu- | (1) |
| doxorubicin | |
| PEG₂₀₀₀-(DSer)4-Ala-Leu-Ala-Leu-doxorubicin | (2) |
| PEG₂₀₀₀-(DSer)-beta-Ala-Leu-Ala-Leu-doxorubicin | (3) |
| PEG₂₀₀₀-beta-Ala-Leu-Ala-Leu-doxorubicin | (4) |
| PEG₂₀₀₀-Ala-Leu-Ala-Leu-doxorubicin | (5) |

Compounds 1, 3 and 4 were synthesized according to method “A,” and compounds 2 and 5 according to method “B.”

Note: The Ala-Leu-Ala-Leu-doxorubicin and beta-Ala-Leu-Ala-Leu-doxorubicin prodrugs were described in International Publication Number WO 96/05863.

2.1) Synthesis Method “A”

The principle of this method is the PEGylation in solution of an NH₂-peptide-doxorubicin compound. 1.5 molar equivalents of mPEG₂₀₀₀-SPA (mPEG₂₀₀₀-succinimidyl propionate ester) (Nektar) dissolved in the chloroform/methanol mixture (4:1) and 5 μl of diisopropylethylamine (DIPEA) was added to the NH₂-peptide-doxorubicin product (obtained in advance after three stages of synthesis, see FIG. 1) that was dissolved in a chloroform/methanol (4:1) mixture.

After about 48 to 72 hours, the final product of the reaction was analyzed by HPLC (High Performance Liquid Chromatography) (see paragraph 2.2 below).

Products (3) and (4) were extracted in dichloromethane at pH 4 (use of lactic acid). The products accumulated in the organic phase. They were then concentrated by evaporation under a vacuum before being freeze-dried.

2.2) Synthesis Method “B”: Peptide Synthesis on a Solid Substrate (SPPS) by Fmoc (Fluoroenyl-Lmethoxycarbonyl) Chemistry

The principle of this method is the synthesis of the PEGylated peptide sequence on a solid polymer substrate (Wang-type resin) and then the coupling between the PEG₂₀₀₀-peptide-OH that is obtained and doxorubicin.

The principle of the SPPS is to bond various amino acids of the expected peptide successively to a solid substrate (Wang-type resin) according to procedures that are known to one skilled in the art (Merrifield, 1963 and 1965; Steward and Young, 1969).

Peptide Synthesis on a Solid Substrate.

The synthetic peptides was obtained by peptide synthesis on a solid substrate in a manual synthesis reactor (AnaSpec) by the chemistry of the Fmoc group, for example. All of the resins that were used (AnaSpec or Novabiochem) during the synthesis on a solid substrate were Wang resins that had the first protected amino acid preattached with an initial substitution provided by the supplier of between 0.4 and 0.7 mmol/g of resin. The Fmoc amino acids were provided by AnaSpec or Novabiochem, with protective groups on the lateral chain such as the following: trityl (Asn, Cys, Gln, and His), Acm (Cys), Boc (Lys and Trp), O-tert-butyl (Asp and Glu), tert-butyl (Ser, Thr, Tyr) and Pbf (Arg). The various polyethylene glycols with the desired molecular weight (PEG-SPA) were obtained from Nektar in the form of a preactivated hydroxysuccinimidyl (OSu) ester. The deprotection of the Fmoc group was carried out by treatment with piperidine in dimethylformamide (DMF). The coupling agent that was used during the synthesis was HCTU (3-oxide of 1H-benzotriazolium 1-[bis(dimethylamino)methylene]-5chloro-hexafluorophosphate (1-)) or HBTU (2-(1H-benzotriazol-1-yl)-1,-1,-3,-3-tetramethyluronium hexafluorophosphate) or HATU (O-(7-azabenzotriazol-1-yl)-N,-N,-N′,-N′-tetramethyluronium hexafluorophosphate) in DMF. Standard coupling cycles of amino acids were carried out as follows: 3×30 seconds, washing with DMF; 3×2 minutes, then 1×7 minutes, deprotection by piperidine that was diluted to 20% in DMF; 5×20 seconds, washing with DMF; 2×15 minutes of coupling with amino acid (2 equivalents), the coupling agent (2 equivalents) and DIPEA (4 equivalents) followed by 3×30 seconds of washing with DMF. The reduction of the substitution of the initial commercial resin was carried out by adding 0.5 to 0.3 molar equivalents of the first Fmoc-AA-OH to be coupled according to the desired sequence (final desired substitution on the order of 0.1 to 0.22 mmol/g) followed by the acetylation of the remaining amino groups (Virender et al., 1981). The coupling of each amino acid was verified by a Kaiser test that is well known to one skilled in the art (Kaiser et al. 1970). After all the amino acids were added according to the desired sequence, the PEGylation on the Wang solid substrate was obtained by 2×2 days of coupling of the active ester PEG-SPA form (Nektar) and DIPEA.

The chemical cleavage of the covalent bond that attaches the PEGylated peptide to the Wang solid substrate was obtained with a mixture that consists of trifluoroacetic acid (TFA):water:triisopropylsilane (TIS) in 95:2.5:2.5 proportions, added to the quite dry resin in the manual synthesis reactor for three hours. The PEGylated pepide was precipitated in cold ether, centrifuged, and washed with ether before being freeze-dried.

Analysis by HPLC

The peptide was analyzed by HPLC with a VYDAC-type column (reverse phase) (C8, 5 μm, 250×4.6 mm), with a first solvent (solvent A) that consists of 0.1% trifluoroacetic acid (TFA) in water and a second solvent (solvent B) that consists of 0.1% TFA in acetonitrile (ACN), with a gradient of 0% to 70% of solvent B in 25 minutes.

2.3) Synthesis Examples: Syntheses of PEG₂₀₀₀-(DSer)₄-Ala-Leu-Ala-Leu-Doxorubicin (2) and PEG₂₀₀₀-Ala-Leu-Ala-Leu-Doxorubicin (5)

1.2 molar equivalent of doxorubicin, HCl dissolved in DMF and 3.2 molar equivalents of DIPEA were added to PEG₂₀₀₀-peptide-OH (PEG₂₀₀₀-(DSer)₄-Ala-Leu-Ala-Leu-OH or PEG₂₀₀₀-Ala-Leu-Ala-Leu-OH) obtained by solid-phase synthesis. The mixture was protected from light and stirred for 15 minutes at ambient temperature before the addition of 1.3 molar equivalents of HATU (coupling agent, PE Biosystem, Foster City, Calif., USA). The reaction was followed by HPLC analysis (Item 2.3 above) for about 3 hours. When the reaction was complete, the solvent was evaporated under vacuum. The residual product was taken up in the water and extracted with dichloromethane. Product (2) preferably accumulates in the organic phase while product (5) is primarily recovered in the aqueous phase. The organic phase that contains product (2) was concentrated by evaporation under a vacuum, and the product was precipitated in ether at ambient temperature. The precipitate that is obtained is dissolved in water before being freeze-dried. The aqueous phase that contains product 5 is collected, frozen in an acetone and solid carbon dioxide bath and freeze-dried. Before freeze-drying, the products were analyzed by HPLC (see Item 2.2 above).

### Example 1C

### Preparation of Product Solutions for Cell Cultures

The products that were used for the experiments of in vitro cell culture were doxorubicin, beta-Ala-Leu-Ala-Leu-doxorubicin, PEG₂₀₀₀-(DSer)₄-beta-Ala-Leu-Ala-Leu-Dox. (1), PEG₂₀₀₀-(DSer)-beta-Ala-Leu-Ala-Leu-Dox. (3), PEG₂₀₀₀-beta-Ala-Leu-Ala-Leu-Dox (4) and PEG₂₀₀₀-Ala-Leu-Ala-Leu-Dox. (5).

The products were dissolved in a minimum volume of water and sterilized by filtration (pore size 0.22 μm). The concentration of solutions was determined by measuring the absorbance (determination at 495 nm based on the molar extinction coefficient of doxorubicin ε=10837 M-1 cm-1).

### Example 1D

### Cell Cultures

The culture medium that was used for the tests that is described below is RPMI 1640 with Glutamax-1 that contains 10% fetal calf serum (serum containing medium) (FCS; Gibco-BRL). (Carlsbad, Calif.).

### Example 1E

### Cytotoxicity Tests

The cytotoxicity tests of doxorubicin and derivatives were carried out with MCF-7/6 cells and LNCaP cells.

The cells were collected by trypsinization, counted, and then inoculated in 96-well plates in 200 μl of serum-containing medium.

The cells were incubated for 24 hours at 37° C. before the addition of the products to be tested:

| Doxorubicin, | |
|---|---|
| Beta-Ala-Leu-Ala-Leu-Dox, | |
| PEG₂₀₀₀-Ala-Leu-Ala-Leu-Dox, | (5), |
| PEG₂₀₀₀-beta-Ala-Leu-Ala-Leu-Dox, | (4), |
| PEG₂₀₀₀-DSer-beta-Ala-Leu-Ala-Leu-Dox, | (3), |
| PEG₂₀₀₀-(DSer)₄-beta-Ala-Leu-Ala-Leu-Dox | (1) |

diluted to various concentrations in the serum-containing culture medium. After an incubation of 48 hours in the presence of various compounds, the cells were finally post-incubated at 37° C. for 24 hours in the presence of drug-free serum-containing medium. After this incubation period, a cell viability test was carried out (WST-1, Roche Molecular Biochemical, Mannheim, Germany).

### Example 1F

### Stability and Hydrolysis Tests of PEGylated Prodrug Derivatives of Doxorubicin

6.1) Effect of PEGylation on the Reactivation of a Prodrug of Doxorubicin, beta-ALAL-Dox, by Peptidases that are Secreted by Tumor Cells

Sub-confluent cultures of tumor cells (MCF-7/6, LS-174T, LNCaP) were washed twice with a saline phosphate buffer solution, and fresh culture medium without serum that contains 0.02% bovine serum albumin (100 μl/cm²) was added. After 24 hours of incubation, the conditioned medium was recovered, centrifuged for 10 minutes at 300 g, buffered with 1 M Tris-HCl, pH 7.5 (1 volume of buffer+19 volumes of medium) and concentrated 20 times by ultrafiltration (cutoff threshold of 10 kDa)(Tris: trishydroxy-methyl-aminomethane).

The reactivation of the PEGylated peptide prodrug derivatives of doxorubicin by the peptidases that are secreted by the tumor cells was compared to that of the initial prodrug, beta-ALAL-Dox, which is hydrolyzed into Leu-Dox and doxorubicin during incubation in the medium that is conditioned by tumor cells. The compounds were diluted to 20 μM in the conditioned medium and incubated for 6 to 18 hours at 37° C. in a thermostated water bath. The hydrolysis of the compounds was quantified by HPLC analysis (see Item 7.1 below).

The following PEGylated prodrugs have been used:

PEG₃₅₀-beta-ALAL-Dox
PEG₇₅₀-beta-ALAL-Dox
PEG₂₀₀₀-beta-ALAL-Dox
PEG₅₀₀₀-beta-ALAL-Dox

6.2) Stability in the Culture Medium

PEG₂₀₀₀-(DSer)₄-beta-Ala-Leu-Ala-Leu-Dox (1), PEG₂₀₀₀-beta-Ala-Leu-Ala-Leu-Dox. (4) and beta-Ala-Leu-Ala-Leu-Dox (control) were diluted to 20 μM in 500 μl of serum-containing medium. The samples were distributed in 24-well plates and incubated at 37° C. in a water-saturated atmosphere that contains 5% CO₂. For each product, an extraction (for extraction method with acetonitrile, see Item 7.3 below) was carried out in triplicate at time 0 and after 1, 2, 6 and 24 hours of incubation. The samples were then analyzed by HPLC (see Item 7.2 below).

6.3) Stability in the Whole Human Blood

PEG₂₀₀₀-(DSer)₄-beta-Ala-Leu-Ala-Leu-doxorubicin (1), PEG₂₀₀₀-beta-Ala-Leu-Ala-Leu-Dox (4), beta-Ala-Leu-Ala-Leu-Dox and Ala-Leu-Ala-Leu-Dox were diluted to 20 μM in 500 μl of whole human blood (kept at 4° C. in a 5 ml sterile citrate tube after collection). The samples were incubated at 37° C. An extraction (for extraction method with acetonitrile, see Item 7.3 below) of each product was carried out in triplicate at time 0 and after 1, 2, 4 and 6 hours of incubation.

### Example 1G

### Analytical Methods

7.1) Basic Extraction

25 μl of the sample to be extracted, 500 μl of water and 100 μl of internal standard (prolyl-daunorubicin at 3.45 μM) were added into tubes containing 2.5 ml of chloroform/methanol mixture (4:1). After alkaline buffer (600 μl of 0.5 M borate buffer, pH 9.8) was added, each tube was stirred immediately for 5 seconds and then centrifuged for 10 minutes to 1800 g.

The organic phase was recovered and dried under a stream of air. The samples were dissolved by adding 500 μl of a mixture that contains 30% acetonitrile and 10% ammonium formate buffer (pH 4). The samples were then filtered and analyzed by HPLC (see Item 7.3 below).

7.2) Extraction with Acetonitrile

10 μl of internal standard (prolyl-daunorubicin) and 50 μM and 150 μl of acetonitrile were added into tubes containing 50 μl of the sample to be extracted. The tubes were stirred and centrifuged for 10 minutes to 13000 g. The supernatant was diluted 4 times in a formate buffer, pH 4, centrifuged, then analyzed by HPLC (see Item 7.3 below).

7.3) HPLC Analysis

All the samples of the products (PEG₂₀₀₀-beta-Ala-Leu-Ala-Leu-Dox. (4), PEG₂₀₀₀-(DSer)₄-beta-Ala-Leu-Ala-Leu-Dox (1), PEG₂₀₀₀-Ala-Leu-Ala-Leu-Dox (5), beta-Ala-Leu-Ala-Leu-Dox and Ala-Leu-Ala-Leu-Dox) were analyzed by HPLC by using the HP1100 system (Agilent Technologies, Palo Alto, Calif. USA) with a column (reverse phaser) such as VYDAC (C8, 5 μm, 250×4.6 mm). Doxorubicin and its derivatives were detected by fluorescence (Lambda_{exc.}=235 nm; Lambda_{em.}=560 nm) and were identified thanks to their respective relative retention time relative to an internal standard. Their concentrations were calculated by taking as a basis the integrated surface of their peaks on the HPLC chromatograms.

The stability curves that were obtained (variations of the concentration, based on time) made it possible to evaluate the stability of the products, and in the case of hydrolysis tests, the amount of Leu-Dox and Dox generated after 6 hours of incubation made it possible to evaluate the hydrolysis of PEGylated derivatives by the enzymes that are released by the tumor cells.

### Example 1H

### In Vivo Study of the Tumor Reactivation of PEGylated Derivatives of a Prodrug of Doxorubicin

The tumor reactivation of PEGylated derivatives of a prodrug of doxorubicin (Suc-beta-ALAL-Dox) was evaluated in athmic mice carrying LS-174T human colon cancer xenografts. The following PEGylated derivatives were used: PEG₂₀₀₀-beta-ALAL-Dox and PEG₂₀₀₀₀-beta-ALAL-Dox.

The mice received an i.v. bolus (intravenous) injection of various compounds at a dose of 69 μmol/kg. The animals were sacrificed 2 and 72 hours after the injection, and the active molecules (Leu-Dox+Dox) accumulated in the tumor were quantified by HPLC (see Item 7.3 above).

### Example 1I

### Chemotherapy Studies of PEGylated Derivatives of a Prodrug of Doxorubicin in Subcutaneous, Ectopic Tumor Xenograft Models

The experimental chemotherapy studies were carried out on athymic mice that carried LS-174T or HCT 116 (colon carcinomas) tumors of human origin previously grafted subcutaneously.

The mice were selected and distributed into the different groups of the study so as to have equal distributions of tumor volumes. The different treatments were randomly assigned to the groups so formed.

Treatments were administered intravenously in the caudal vein. During these injections, the animals received a constant volume of 10 μl/g of product (Workman et al., 1998). After the injection, the clinical signs were duly followed (each hour during the first day, and daily until the end of the study), as well as the weight that was measured at the same time as the tumor volume (mm³). The growth of the tumor was monitored 2× per week by measuring, with the aid of a caliper, two perpendicular diameters (or “diagonals”) of the tumor (the largest and the smallest “diagonal”).

To determine the variations of the tumor volume of the different groups, the median of the relative tumor volumes (RTV for “relative tumor volume”) that express in percent the variations of the medians of the tumor volumes relative to day 0 was calculated. To estimate the effect of the various treatments, the inhibition of the growth was determined by calculating the ratio between the median of the RTV of the treated groups (T) and that of control group (C) that is expressed by percentage (T/C(%)).

The lowest value of the T/C ratio was used as a parameter to estimate the maximum efficacy of the products that were evaluated.

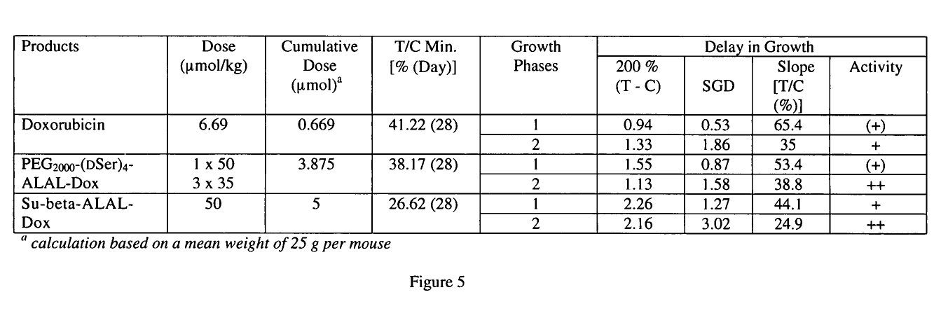

The period of inhibition of the growth of the tumor was evaluated by calculating the growth delay between the treated groups and the control group (T-C) for one doubling (DT for “Doubling Time”) of the RTV medians (200% indication in FIG. 5 representing the doubling time of the control group). The Specific Growth Delay (SGD) was also calculated as (DT_{treated}−DT_{control})/(DT_{control}).

The activity of the different products tested was evaluated by taking as a basis the criteria recommended by the EORTC (European Organization for Research and Treatment of Cancer) (Boven et al., 1992; Langdon et al., 1994) and presented in Table I. The method of estimating the efficacy of the compounds, however, was adapted, since the criteria proposed in the literature (Boven et al., 1992) are valid only for tumors that exhibit a monophase linear growth.

The LS-174T tumor that was used was characterized by a 2-phase growth, whereby the terminal phase (phase 2) was much faster than the first phase (phase 1). For these reasons, growth delays (T-C) and specific growth delays (SGD) were calculated from linear regressions of different phases that characterize the growth of the tumors.

Table 1 below shows the evaluation of the effectiveness of a chemotherapy agent according to the criteria recommended by the EORTC (Boven et al., 1992; Langdon et al., 1994). Level of activity: −=no activity; (+)=very low activity; +=low activity; ++=moderate activity; +++=significant activity; ++++=very significant activity.

TABLE 1

| Criteria of the EORTC | Activity |
|---|---|
| SGD < 1.0 and T/C > 50% | − |
| SGD > 1.0 or T/C < 50% | (+) |
| SGD > 1.0 and T/C < 50% | + |
| SGD > 1.5 and T/C < 40% | ++ |
| SGD > 2.0 and T/C < 25% | +++ |
| SGD > 3.0 and T/C < 10% | ++++ |

### Example 1J

### Chemotherapy Study with the PEGylated Derivatives of TNF-Alpha in an Intradermal Ectopic Xenograft Model

10.1) Cell Line

B16-BL6 cells: murine melanoma cells selected in vivo for their invasiveness of the bladder and that have a high capacity for spontaneously forming pulmonary metastases were used. These cells were obtained from the “NCI-Frederick Cancer Research and Development Center,” Maryland, USA.

10.2) Anti-Cancer Agents for the Chemotherapy Study

RhTNF-α (“Recombinant human Tumor Necrosis Factor alpha”) was manufactured by Henogen (Gosselies, Belgium).

The (PEG₅₀₀₀)n-TNFα (PEG₅₀₀₀-Ala-Leu-Ala-Leu)n-TNFα and (PEG₅₀₀₀-(DSer)₄-Ala-Leu-Ala-Leu)n-TNFα conjugates were synthesized. “n” varies from 1 to 18 (with 18 being the total lysine number per TNFα molecule in its trimeric form, knowing that each TNF monomer comprises 6 lysine residues). The synthesis of the (PEG-peptide)ₙ-TNFα derivatives took place as follows: the peptide was synthesized on solid phase, then coupled with an activated (N-hydroxysuccinimide) polyethylene glycol (PEG) in the presence of dimethylformamide (DMF) and diisopropylethylamine (DIPEA). After purification, the PEG-peptide-OH that was dissolved in DMF was activated in the presence of a coupling agent that is well known to one skilled in the art (ex: TSTU, 2-succinimido-1,1,3,3-tetramethyluronium tetrafluoroborate). The rhTNFα that was solubilized in 20 mmol of phosphate buffer, pH 7.4, containing 1% mannitol, was then added, and the reaction mixture was stirred at ambient temperature for one night. The coupling product was recovered, and excess PEG-peptide-OH was eliminated by ultracentrifugation. The final product concentration was determined by a protein assay.

10.3) The Animals

Male C57BL/6J mice (5 weeks after delivery by the Charles River Laboratories, France) were used.

Handling of the animals was in accordance with the recommendations of the UKCCCR (United Kingdom Coordinating Committee on Cancer Research; Workman et al., 1998) and the FELASA (Federation of European Laboratory Animal Science Associations; Nicklas et al., 2002; Rehbinder et al., 2000; Rehbinder et al., 1996) on the use and the well-being of animals in experimental chemotherapy studies.

The experimental chemotherapy study was carried out using C57BL6J male mice carrying B16-BL6 murine tumors (melanoma) that were previously grafted intradermally.

The mice were selected and distributed into the various groups of the study so as to have equal distributions of tumor volumes. The different treatments were randomly assigned to groups that were formed.

The treatments were administered intravenously on days 0 and 3. During these injections, the animals received a constant volume of 10 μl/g of product (Workman et al., 1998). After the injection, the clinical signs were duly recorded (each hour during the first day, and daily until the end of the study), as well as the weight that was measured at the same time as the tumor volume. The growth of the tumor was monitored 2× per week by measuring two perpendicular diameters (or “diagonals”) of the tumor (the largest and the smallest “diagonal”) using a caliper.

The anti-tumor efficacy was quantified by the value of the T/C ratio. The determination method is described in detail in Item 9 above.

### Example 2

### Effect of PEGylation on the Reactivation of a Prodrug of Doxorubicin, beta-ALAL-Dox, by Peptidases that are Secreted by the Tumor Cells

The compounds beta-ALAL-Dox, PEG₃₅₀-beta-ALAL-Dox, PEG₇₅₀-beta-ALAL-Dox, PEG₂₀₀₀-beta-ALAL-Dox and PEG₅₀₀₀-beta-ALAL-Dox (20 μM) were incubated for 6 hours in a conditioned medium of MCF-7/6 or LS-174T tumor cells. The reactivation of the prodrugs (release of doxorubicin and Leu-Dox) was quantified by HPLC.

Table 2 below shows the hydrolysis of the prodrug beta-ALAL-Dox and its PEGylated derivatives. The results represent the concentrations of doxorubicin (Dox) and Leucyl-doxorubicin (L-Dox or Leu-Dox) reached after 6 hours and are expressed as the mean value obtained from three independent experiments±standard deviation (n=9).

TABLE 2

| Cell Types | |
|---|---|
| MCF-7/6 | LS-174T |

| Tested | DOX | L-DOX | DOX | L-DOX |
|---|---|---|---|---|
| Compounds | (μM) | (μM) | (μM) | (μM) |
| beta-ALAL- | 1.45 ± | 8.14 ± | 0.22 ± | 5.26 ± |
| Dox | 0.07 | 0.27 | 0.02 | 0.09 |
| PEG₃₅₀-beta- | 0.62 ± | 3.29 ± | 0.00 | 2.59 ± |
| ALAL-Dox | 0.02 | 0.28 | | 0.14 |
| PEG₇₅₀-beta- | 0.19 ± | 1.62 ± | 0.00 | 2.00 ± |
| ALAL-Dox | 0.02 | 0.03 | | 0.12 |
| PEG₂₀₀₀-beta- | 0.00 | 0.69 + | 0.00 | 1.31 ± |
| ALAL-Dox | | 0.45 | | 0.21 |
| PEG₅₀₀₀-beta- | 0.00 | 0.66 ± | 0.00 | 0.34 ± |
| ALAL-Dox | | 0.45 | | 0.05 |

The results that are presented in the table above show that the reactivation of prodrugs and therefore the release of active molecules (Leu-Dox+doxorubicin) is inhibited especially as the PEG molecular weight increases. The PEGylation of the prodrug of doxorubicin (beta-ALAL-Dox) prevents its reactivation by tumor peptidases. This inhibition is correlated with the size of the PEG. The inventors thus conceived the hypothesis that this inhibition is probably the consequence of a steric hindrance phenomenon that limits the accessibility of the cleavage site.

### Example 3

### In Vivo Study of the Tumor Reactivation of PEGylated Derivatives of a Prodrug of Doxorubicin

The in vivo tumor reactivation of PEGylated derivatives of a prodrug of the doxorubicin (beta-ALAL-Dox) was evaluated using athymic mice carrying LS-174T human colon cancer tumor xenografts. Table 3 below shows the average (obtained in 18 mice) of Dox and Leu-Dox concentrations accumulated in tumors at 2 and 72 hours after the injection of the tested compound.

TABLE 3

| | Concentrations (nmol/mg) with | |
|---|---|---|
| | Regard to LS-174T | |
| | 2 Hours After | 72 Hours After |
| Tested | Injection | Injection |

| Compounds | DOX | Leu-DOX | DOX | Leu-DOX |
|---|---|---|---|---|
| PEG₂₀₀₀-beta- | 0.38 ± | 0.78 ± | 1.71 ± | 0.12 ± |
| ALAL-Dox | 0.14 | 0.28 | 0.5 | 0.07 |
| PEG₂₀₀₀₀-beta- | 0.60 ± | 0.66 ± | 0.76 ± | 0.03 ± |
| ALAL-Dox | 0.09 | 0.05 | 0.14 | 0.02 |
| Suc-beta- | 1.93 ± | 4.64 ± | 5.41 ± | 0.05 ± |
| ALAL-Dox | 0.50 | 0.50 | 0.24 | 0.02 |

The table above shows that in both cases, PEG₂₀₀₀-beta-ALAL-Dox and PEG₂₀₀₀₀-beta-ALAL-Dox are less reactivated in Dox and Leu-Dox than the control Suc-beta-ALAL-Dox. The PEGylation of this prodrug of doxorubicin therefore rigorously reduces the reactivation in tumors.

### Example 4

### Effect of the Insertion of a Spacer on the Reactivation of PEGylated Prodrugs of Doxorubicin by the Peptidases Secreted by Tumor Cells

Beta-ALAL-Dox prodrugs (20 μM) were incubated for 6 to 18 hours in a culture medium conditioned by LS-174T or LNCaP tumor cells. The reactivation of the prodrugs (release of doxorubicin and Leu-Dox) was measured by HPLC.

Two spacers were tested:
Four hydrophilic DSerine ((DSer)₄) residues
Insertion of 2 or 3 6-aminohexanoic acid residues (or aminocaproic acid, that is a hydrophobic amino acid longer than serine), denoted Ahx.

Table 4 below shows the hydrolysis of the Suc-beta-ALAL-Dox (control) prodrug and the PEGylated beta-ALAL-Dox derivatives. The results represent the percentages of doxorubicin and Leu-doxorubicin that were released relative to the control (n=3).

TABLE 4

| | Cell Types | |
|---|---|---|
| | LS-174T | LNCaP |
| | Dox + | Dox + |
| Tested Compounds | Leu-Dox (%) | Leu-Dox (%) |
| Suc-beta-ALAL-Dox | 100.00 ± 1.18 | 100 |
| PEG₂₀₀₀-beta-ALAL-Dox | 11.52 ± 1.15 | 32 |
| PEG₂₀₀₀-(Ahx)₃-beta-ALAL-Dox | 10.14 ± 0.69 | Not Tested |
| PEG₂₀₀₀-(DSer)₄-beta-ALAL-Dox | 22.35 ± 1.61 | 66 |
| PEG₅₀₀₀-beta-ALAL-Dox | 1.98 ± 0.29 | Not Tested |
| PEG₅₀₀₀-Ahx-beta-ALAL-Dox | 3.07 ± 0.20 | Not Tested |
| PEG₅₀₀₀-(Ahx)₂-beta-ALAL-Dox | 1.29 ± 0.49 | Not Tested |
| PEG₂₀₀₀₀-beta-ALAL-Dox | 0.98 ± 0.40 | Not Tested |
| PEG₂₀₀₀₀-(Ahx)-beta-ALAL-Dox | 0.49 ± 0.20 | Not Tested |
| PEG₂₀₀₀₀-(Ahx)₂-beta-ALAL-Dox | 1.43 ± 0.34 | Not Tested |

The results of the table above show again that the hydrolysis of the PEGylated derivatives decreases significantly relative to that of Suc-beta-ALAL-Dox.

The insertion of four DSeine residues between PEG and beta-ALAL-Dox increased the release of doxorubicin and Leu-Dox 2-fold relative to the PEG₂₀₀₀ derivatives without a spacer or with a 3 Ahx residue hydrophobic spacer, following incubation of the compounds in the conditioned medium of LS-174T or LNCaP cells.

The insertion of aminohexanoic acid residues (hydrophobic amino acid) did not increase the reactivation of PEGylated derivatives of the prodrug relative to the derivatives without a spacer.

### Example 5

### Cytotoxicity Tests of PEGylated Derivatives of a Prodrug of Doxorubicin

MCF-7/6 tumor cells were incubated for 48 hours in serum-containing medium that contained growing concentrations of Doxorubicin, beta-ALAL-Dox, PEG₂₀₀₀-beta-ALAL-Dox, PEG₂₀₀₀-DSer-beta-ALAL-Dox or PEG₂₀₀₀-(DSer)₄-beta-ALAL-Dox. They were then post-incubated for 24 hours in drug-free serum containing medium. The cytotoxicity of the products was estimated by a cell viability test (WST-1, Roche Molecular Diagnostic, Mannheim, Germany).

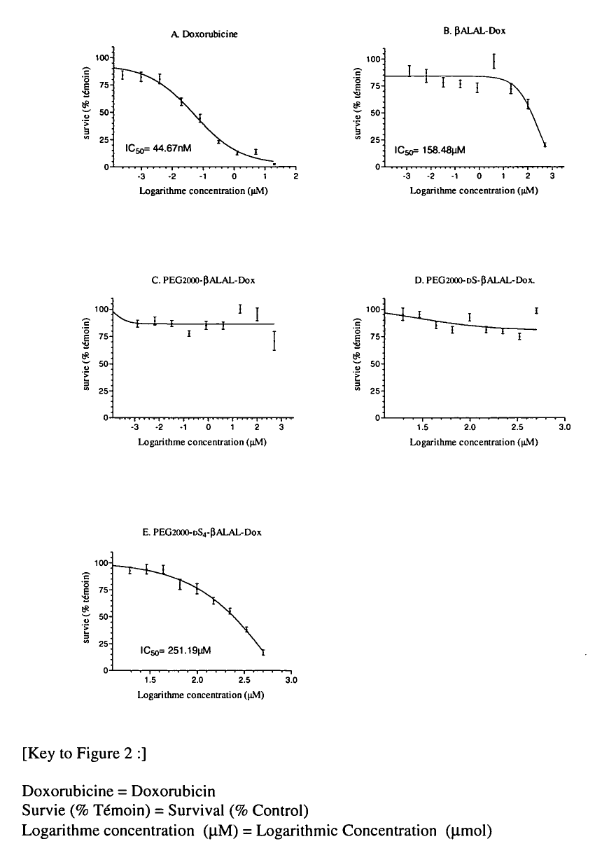

The results of 3 independent experiments are presented in FIG. 2.

The IC₅₀ values (50% inhibiting concentration) of doxorubicin and beta-ALAL-Dox were respectively 0.045 μM and 158.48 μM, which confirms the prodrug nature of the beta-ALAL-Dox. The comparison of the IC₅₀ mean values that were obtained for beta-ALAL-Dox and PEG₂₀₀₀-beta-ALAL-Dox indicate that PEGylation inhibits the reactivation of the prodrug since PEG₂₀₀₀-beta-ALAL-Dox is not cytotoxic up to a concentration of 500 μM. The prodrug that comprises a DSer between the PEG and beta-ALAL-Dox (PEG₂₀₀₀-DSer-beta-ALAL-Dox) was similarly not cytotoxic. By contrast, the prodrug that comprises the molecular spacer (DSer)₄ between PEG and beta-ALAL-Dox, (PEG₂₀₀₀-(DSer)₄-beta-ALAL-Dox) was cytotoxic with an activity (IC₅₀=251.19 μM) similar to that of the non-PEGylated prodrug, beta-ALAL-Dox.

These results show that the insertion of 4 DSerine residues between PEG and beta-ALAL-Dox allows the reactivation of the PEGylated prodrug. By contrast, the PEGylated prodrug without a spacer cannot be reactivated. The toxicity of this latter prodrug results primarily from its extra-blood reactivation, i.e., from the better release of doxorubicin relative to the other tested compounds.

### Example 6

### Efficacy Study of a PEGylated Derivative of a Prodrug of Doxorubicin, beta-ALAL-Dox, in a LS-174T Human Colon Carcinoma Xenograft Model

The anti-tumor activity of doxorubicin, succinyl-beta-Ala-Leu-Ala-Leu-Dox, and PEG₂₀₀₀-(DSer)₄-Ala-Leu-Ala-Leu-Dox was tested in athymic mice (nude/nude NMRI) carrying ectopic xenografts of an LS-174T human tumor. The products were injected intravenously (10 μl/g, 6 mice per group).

PEG₂₀₀₀-(DSer)₄-ALAL-Dox was administered initially by i.v. bolus at doses of 50 μmol/kg and 45 μmol/kg. One death was observed with the two administered doses.

The administered doses were reduced starting from day 7. PEG₂₀₀₀-(DSer)₄-ALAL-Dox was injected at 35 μmol/kg and 25 μmol/kg (once a week, 3 injections). The other compounds were administered by i.v. bolus injection on days 0, 7, 14 and 21 (doxorubicin 6.69 and 8.6 μmol/kg, Suc-beta-ALAL-Dox 45 and 50 μmol/kg).

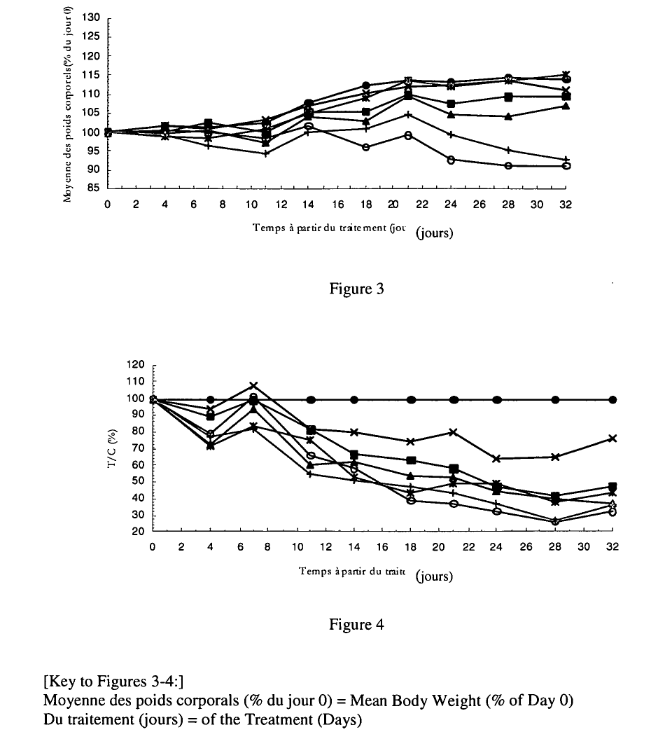

FIG. 3 shows the variations of the mean body weight (percent of the value at beginning of the weekly treatment.) As of the second week, no significant weight loss was observed in the groups treated with doxorubicin and with PEG₂₀₀₀-(DSer)₄-ALAL-Dox. A slight weight loss was observed in the groups treated with Suc-beta-ALAL-Dox. In this latter case, the maximum weight loss was 10% on day 32 in the two treated groups.

FIG. 4 shows the variations of the median relative tumor volumes (RTV) of the groups of treated mice (T) relative to controls (C) (NaCl). Although administered at doses below the maximum tolerated dose (MTD), all the tested products had an anti-tumor activity.

During the treatment, two tumor growth phases were observed (FIG. 5). During the first growth phase, the activity of PEG₂₀₀₀-(DSer)₄-ALAL-Dox (1×50+3×35 82 mol/kg) was comparable to that of doxorubicin (6.69 μmol/kg) and was lower than that of succinyl-beta-ALAL-Dox (50 μmol/kg). By contrast, during the second growth phase, PEG₂₀₀₀-(DSer)₄-ALAL-Dox was more active than doxorubicin and as active as Suc-beta-ALAL-Dox. Overall, it was observed that the Suc-beta-ALAL-Dox products (T/C min=26.6%; specific growth delay (SGD=3.02) and PEG₂₀₀₀-(DSer)₄-ALAL-Dox (T/C min=38%; SGD=1,58) were more active than the doxorubicin.

### Example 7

### Stability of PEGylated Derivatives of Prodrugs of Doxorubicin in the Serum-Containing Medium

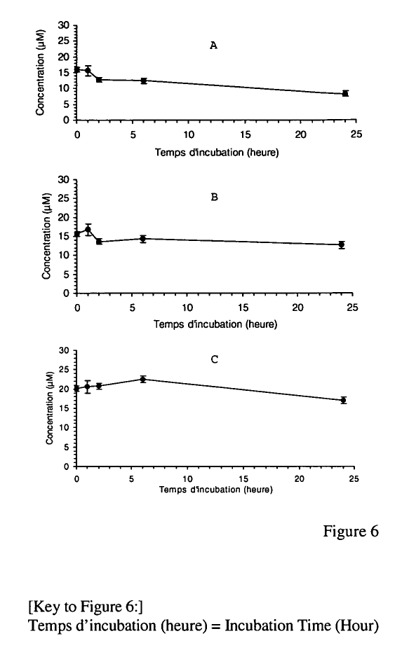

The stability of the beta-ALAL-Dox, PEG₂₀₀₀-beta-ALAL-Dox and PEG₂₀₀₀-(DSer)₄-beta-ALAL-Dox compounds was evaluated after a 24-hour incubation at 37° C. in culture medium containing 10% fetal calf serum. The concentrations of initial products and the possible metabolites formed were quantified by HPLC. The 3 tested conjugates were stable. No degradation product was detected (FIG. 6).

### Example 8

### Stability of PEGylated Derivatives of Prodrugs of Doxorubicin in the Whole Human Blood.

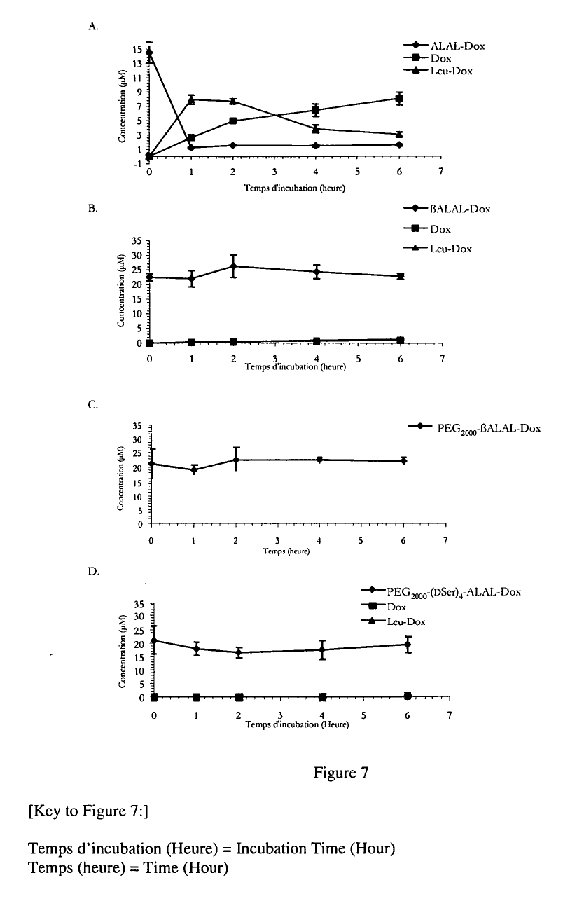

The beta-ALAL-Dox, PEG₂₀₀₀-ALAL-Dox, PEG₂₀₀₀-beta-ALAL-Dox and PEG₂₀₀₀-(DSer)₄-ALAL-Dox compounds were diluted to 20 μM in citrated human blood and incubated at 37° C. At different time points, the concentrations of the conjugates and possible metabolites formed were evaluated by HPLC. FIG. 7 illustrates the variations of the concentration of products based on the incubation time.

The ALAL-Dox compound was used as a control. The latter is not stable in blood. After 1 hour, 80% of the starting product was degraded in Leu-Dox and Dox. The other 3 tested conjugates were stable for at least 6 hours in the whole human blood (FIG. 7). At the time of 6 hours, only a very slight hydrolysis of the beta-ALAL-Dox (8% of Dox and 10% of L-Dox) and PEG₂₀₀₀-(DSer)₄-ALAL-Dox (5% of Dox and 5% of Leu-Dox) was observed. These results confirm that the replacement of the terminal amine of the oligopeptide by a β-alanine prevents the hydrolysis of the sequence by the blood peptidases. The stability of the tested PEGylated derivatives may be due to the presence of amino acids that are not natural (β-alanine or D-serine) but also to the PEG that is used as a stabilizing group.

### Example 9

### Efficacy Study of a PEGylated Derivative of a Prodrug of Doxorubicin, beta-ALAL-Dox, in HCT-116 Human Colon Carcinoma Xenograft Model.

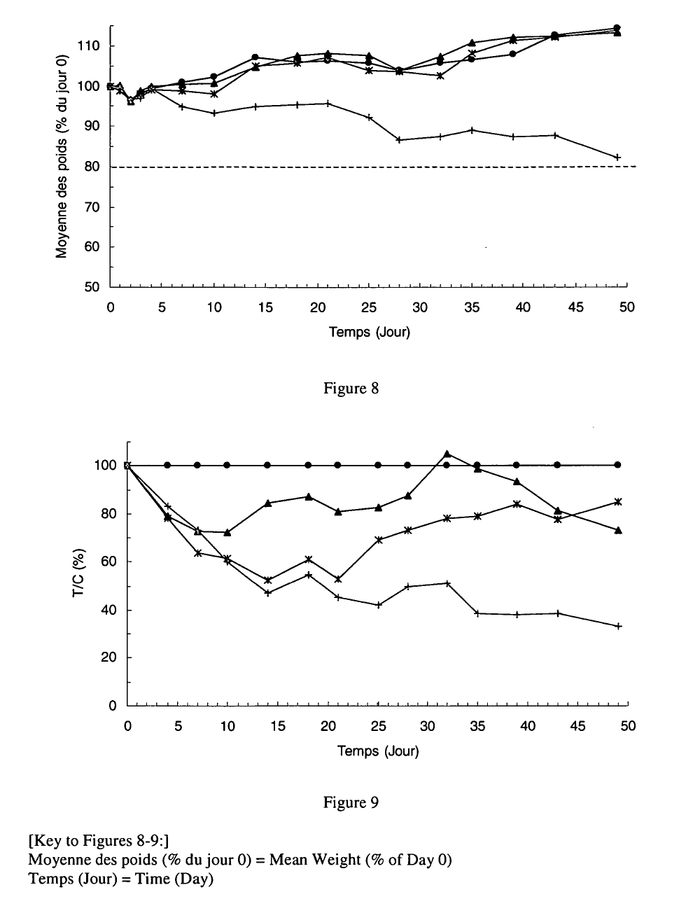

The anti-tumor efficacy of PEG₂₀₀₀-(DSer)₄-ALAL-Dox was compared to that of succinyl-βALAL-Dox in an HCT-116 human colon carcinoma xenograft model that was implanted subcutaneously in Swiss nude/nude mice. The animals received 5 i.v. bolus injections (for 5 consecutive days) of succinyl-βALAL-Dox at 30 μmol/kg or PEG₂₀₀₀-(DSer)₄-ALAL-Dox at 53 and 110 μmol/kg (6 mice/group). No mortality was observed in the treated groups. The results of FIG. 8 show the variations of the body weight of the animals. Regardless of the dose administered, no toxicity of PEG₂₀₀₀-(DSer)₄-ALAL-Dox was observed, while the mice treated with succinyl-βALAL-Dox lost weight (about 20% at the end of the study, day 49), indicating that the maximum tolerated dose (MTD) is reached in this group. The anti-tumor efficacy is shown by the T/C curve that comprises two phases (FIG. 9). In the first (up to day 21), PEG₂₀₀₀-(DSer)₄-ALAL-Dox (110 μmol/kg) had an activity similar to that of succinyl-βALAL-Dox (30 μmol/kg). In the second phase, the activity of the PEGylated derivative (110 μmol/kg) decreased relative to that of succinyl-PALAL-Dox. At 53 μmol/kg, the PEGylated derivative induced a tumor regression for the week of treatment beyond which tumor growth resumed. These results show that PEG₂₀₀₀-(DSer)₄-ALAL-Dox is less toxic than succinyl-βALAL-Dox and is active for the first phase of the study. The MTD of the PEGylated derivative was not reached in this study.

### Example 10

### Chemotherapy Study of PEG2000-(DSer)₄-ALAL-Dox vs PEG₂₀₀₀-ALAL-Dox in an HCT-116 Human Colon Carcinoma Xenograft Model

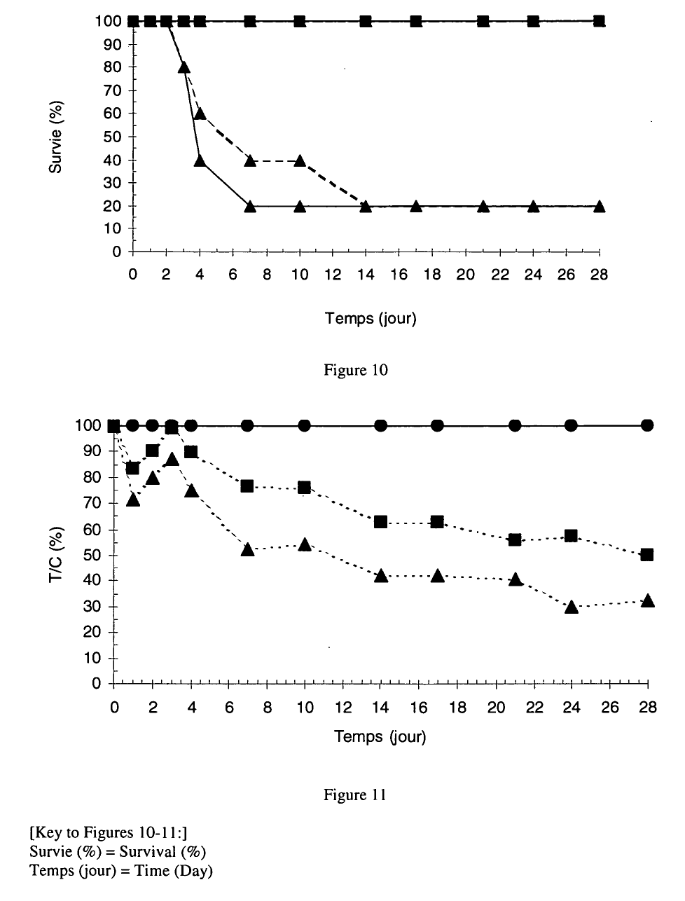

The anti-tumor efficacy of PEG₂₀₀₀-(DSer)₄-ALAL-Dox was compared to that of PEG₂₀₀₀-ALAL-Dox in an HCT-116 human colon carcinoma xenograft model implanted subcutaneously in Swiss nude/nude mice. The animals received 5 i.v. bolus injections (for 5 consecutive days) of the compounds at doses of 200, 300, and 400 μmol/kg (5 mice/group). Contrary to PEG₂₀₀₀-ALAL-Dox, PEG₂₀₀₀-(DSer)₄-ALAL-Dox was toxic at 300 and 400 μmol/kg and induced a weight loss and the death of the animals (FIG. 10). This toxicity suggests a more significant reactivation in the extra-blood compartment of PEG₂₀₀₀-(DSer)₄-ALAL-Dox compared to PEG₂₀₀₀-ALAL-Dox. At one equimolar and non-toxic dose (200 μmol/kg), PEG₂₀₀₀-(DSer)₄-ALAL-Dox had a better anti-tumor efficacy than that of PEG₂₀₀₀-ALAL-Dox (FIG. 11). Again, these results support the hypothesis of a greater tumor reactivation of the compound that comprises the molecular spacer that comprises 4 serines in conformation D. These data clearly demonstrate the positive effect of the insertion of 4 D-serines between PEG and the cleavable sequence.

### Example 11

### Efficacy Study of PEGylated Derivatives of TNF-α

The anti-tumor activity of (PEG₅₀₀₀-(DSer)₄-ALAL)ₙ-TNFα was compared to that of (PEG₅₀₀₀)ₙ-TNFα and (PEG₅₀₀₀-Ala-Leu-Ala-Leu)ₙ-TNFα in C57BL/6J mice carrying B16BL6 murine melanoma (grafted intradermally).

The conjugates were administered intravenously at a dose of 2000 μg eq. TNF-α/kg on days 0 and 3. No immediate sign of toxicity was observed with the administered dose.

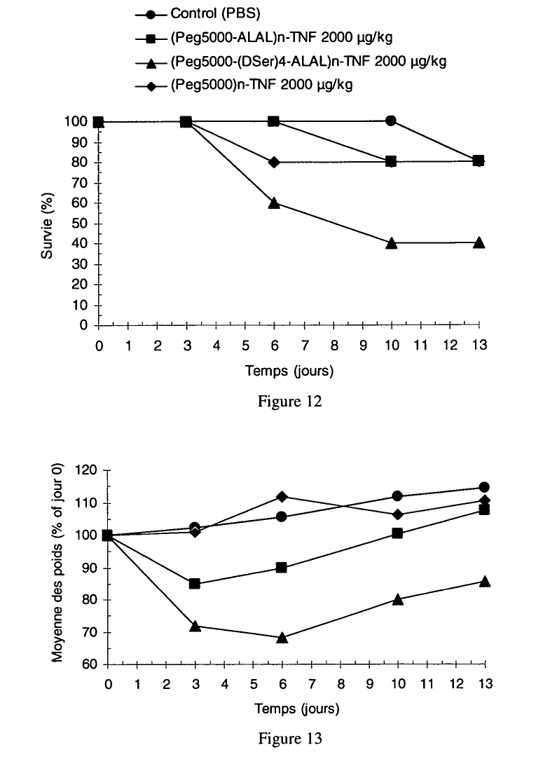

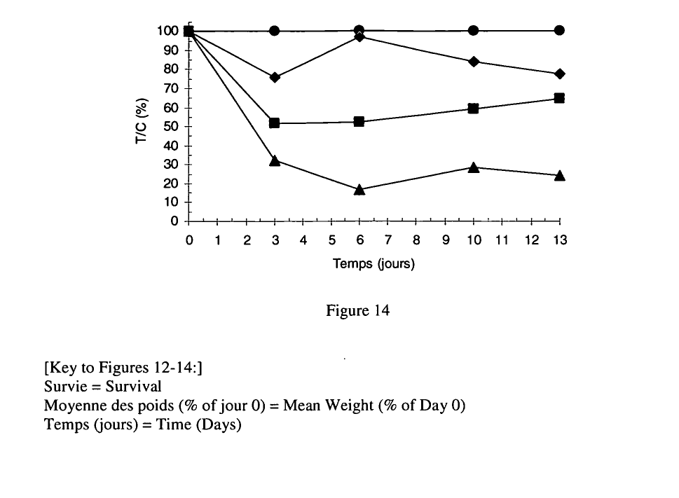

FIG. 12 shows the variations of the survival of the animals. (PEG₅₀₀₀-(DSer)₄-ALAL)ₙ-TNFα resulted in the death of about 40% of the mice on day 6 and 60% on day 10. The mortality that was observed in the other groups, including the control group, did not exceed 20% (1 mouse out of 5) and was probably due to the invasive and metastasic characteristics of the B16-BL6 tumors.

FIG. 13 shows the variations of the mean body weight over time (percent of the value at the beginning of the treatment). The day after the first injection, a significant weight loss was observed in the groups treated with (PEG₅₀₀₀-ALAL)ₙ-TNFα and (PEG₅₀₀₀-(DSer)₄-ALAL)ₙ-TNFα (15 and 28%, respectively). After the second injection, this weight loss was emphasized in the group that was treated with (PEG₅₀₀₀-(DSer)₄-ALAL)ₙ-TNFα. In the latter, the maximum weight loss was 32% on day 6. In contrast, in the group treated with (PEG₅₀₀₀-ALAL)ₙ-TNFα, weight recovery was observed after the second injection (day 3). No weight loss was observed in the group treated with (PEG₅₀₀₀)ₙ-TNFα.

The anti-tumor efficacy of the different compounds is shown in FIG. 14 by the variations of the T/C ratio (ratio between the median of the RTV of treated groups (T) and that of control group (C)). These results clearly show that the activation of the prodrugs of TNFα, necessary to the release of an active form of the protein, varies based on the structure of the conjugates. (PEG₅₀₀₀)ₙ-TNFα was not active, and (PEG₅₀₀₀-ALAL)ₙ-TNFα was less active (T/C min=51.7% (j3); SGD=1.66) than (PEG₅₀₀₀-(DSer)₄-ALAL)ₙ-TNFα. (T/C min=16.7% (j6); SGD=3.72). These results demonstrate a correlation between the activation and the toxicity of the conjugates. The toxicity was low or absent (weight loss) in the absence of a peptide between PEG and TNFα. The insertion of an ALAL bond between these two parts induces a more significant toxicity of the product that reflects its higher reactivation. The extension of this peptide (ALAL) by the insertion of a hydrophilic spacer (DSer)₄ induced a significant increase in the toxicity of the conjugate that very likely results from its increased sensitivity to reactivation.

### Example 12

### Syntheses of PEG_{30,000}-(DSer)₈-Ala-Leu-Ala-Leu-Doxorubicin (1) and PEG_{30,000}-Ala-Leu-Ala-Leu-Doxorubicin (2)

1.2 molar equivalents of doxorubicin, HCl and 1 molar equivalent of Fmoc-peptide-OH (Fmoc-(DSer)₄-Ala-Leu-Ala-Leu-OH for product 1′ or Fmoc-Ala-Leu-Ala-Leu-OH for product 2′ obtained previously by synthesis on solid substrate) are dissolved in DMF. The pH is adjusted to 8 with DIPEA before the addition of 1.2 molar equivalents of HCTU (coupling agent). The mixture is stirred at room temperature and the reaction is followed by HPLC chromatographic analysis. When the reaction is complete, the Fmoc-peptide-Dox is precipitated with ether, recovered by filtration and freeze-dried. The deprotection of Fmoc-Peptide-Dox is carried out in the presence of 50 molar equivalents of piperidine at 10% in DMF (5 minutes) and the reaction is stopped by addition of lactate buffer. The residual product is subjected to purification using C18 ODS-A YMC gel and reverse-phase preparative HPLC before freeze-drying.

For the synthesis of product 1′, 1 molar equivalent of the ester activated PEG_{30,000}-SPA and 1,2 molar equivalent of H-(DSer)₄-OH are dissolved in DMF and the pH is adjusted to 8 with DIPEA. The mixture is incubated for 2 days at room temperature. The residual product is precipitated with ether, dialyzed against water to remove the exceeding peptide and freeze-dried. A MALDI-TOF MS analysis is performed prior to the following stage of the synthesis. The PEG_{30,000}-(DSer)₄-OH (1 molar equivalent) and the H-(DSer)₄-Ala-Leu-Ala-Leu-Dox (1.2 molar equivalents) dissolved in DMF are mixed and the pH is adjusted to 8 with DIPEA before adding HCTU (1.2 molar equivalents). The mixture is stirred at room temperature and the reaction is followed by HPLC chromatographic analysis. When the reaction is complete, the product 1′ is precipitated with ether, recovered by filtration and freeze-dried. The product 1′ is finally purified by reverse-phase preparative HPLC and analyzed by MALDI-TOF MS.

For the synthesis of product 2′, 1 molar equivalent of the ester activated PEG_{30,000}-SPA and 1,2 molar equivalent of H-ALAL-Dox are dissolved in DMF and the pH is adjusted to 8 with DIPEA. The mixture is stirred at room temperature and the reaction is followed by HPLC chromatographic analysis. When the reaction is complete, the product 2′ is precipitated with ether, recovered by filtration and freeze-dried. The product 2′ is finally purified by alkaline extraction or by reverse-phase preparative HPLC and analyzed by MALDI-TOF MS.

### Example 13

### Biodistribution of PEG_{30,000}-(DSer)₈-ALAL-Dox in Comparison with Peg_{30,000}-ALAL-Dox in LS-174T Tumors In Vivo

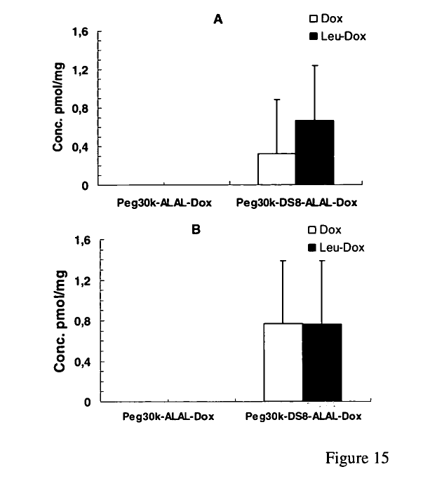

The intratumoral release of Ala-Leu-doxorubicin, Leu-doxorubicin and doxorubicin was compared following intravenous infusion of either PEG_{30,000}-(DSer)₈-ALAL-Dox or PEG_{30,000}-ALAL-Dox in a LS-174T human colorectal adenocarcinomas xenografts model implanted subcutaneously in nude NMRI mice. Animals received either PEG_{30,000}-(DSer)₈-ALAL-Dox or PEG_{30,000}-ALAL-Dox at 80 μmol/kg delivered by a 30 minute infusion. Animals were sacrificed 1, 3, 6 or 24 hours after the end of the infusion (three animals per group) and the tumors were recovered. The level of the parent compound, together with the major metabolites Ala-Leu-doxorubicin, Leu-doxorubicin or doxorubicin present in the tumors were evaluated following acetonitrile extraction by HPLC analysis. The kinetic data for the tumor accumulation of the pegylated compounds and their metabolites is shown in FIG. 15. For the PEG_{30,000}-(DSer)₈-ALAL-Dox molecule (noted in FIG. 15, Peg30k-(DS)₈-ALAL-Dox), the ALAL peptidic sequence is rapidly cleaved in the presence of tumor specific peptidases to yield both Leu-doxorubicin and doxorubicin, which is detectable at the initial time point (one hour following the infusion). For the PEG_{30,000}-ALAL-Dox molecule (noted in FIG. 15, Peg30k-ALAL-Dox), the ALAL is less rapidly cleaved than observed for the PEG_{30,000}-(DSer)₈-ALAL-Dox, as the metabolites Leu-doxorubicin and doxorubicin are not detected until 6 hours after infusion. The level of Ala-Leu-doxorubicin release by both parent molecules was below the detection threshold in this analysis. The AUC₀₋₂₄ₕᵣₛ for the two major metabolites released (Leu-doxorubicin and doxorubicin) shows that the PEG_{30,000}-(DSer)₈-ALAL-Dox molecule (AUC₀₋₂₄ₕᵣₛ Leu-doxorubicin 21.10 UNITS, AUC₀₋₂₄ₕᵣₛ doxorubicin 3.90 UNITS) is a better substrate for cleavage than the Peg_{30,000}-ALAL-Dox molecule (AUC₀₋₂₄ₕᵣₛ Leu-doxorubicin 13.26 UNITS, AUC₀₋₂₄ₕᵣₛ doxorubicin 1.56 UNITS). Both results confirm that PEG_{30,000}-(DSer)₈-ALAL-Dox is a superior substrate for in vivo tumor specific reactivation and that without a (DSer)₈ the reactivation of the PEG_{30,000}-ALAL-Dox prodrug is inhibited.

### Example 14

### In Vitro Reactivation of Ester PEG_{20,000}(DSer)₈-ALAL-Dox in Comparison with Ester PEG_{20,000}-ALAL-Dox in LS-174T Tumor Cell Conditioned Medium

The in vitro reactivation of ester Peg_{20,000}(DSer)₈-ALAL-Dox and ester PEG_{20,000}-ALAL-Dox in tumor cell conditioned medium (containing tumor specific endopeptidases) was undertaken to define which of these molecules represents the best substrate for reactivation and release of metabolites Ala-Leu-doxorubicin, Leu-doxorubicin or doxorubicin.

The monofunctional polymer reagent used is an ester PEG of the succinimidyl succinate of formula I: 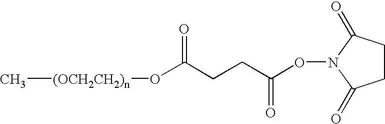
wherein n is about 444

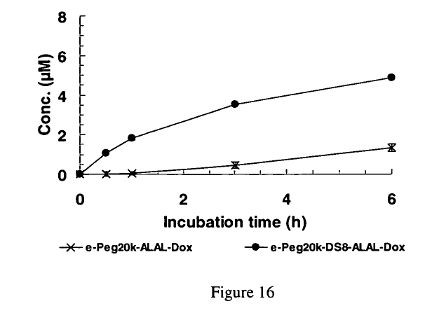

Ester PEG_{20,000}-(DSer)₈-ALAL-Dox or ester PEG_{20,000}-ALAL-Dox were incubated with D-MEM-F12 culture medium that had previously been conditioned by incubation during 24 hours with LS-174T tumor cells at 37° C. The conditioned medium was concentrated 20 times before use. At different post-incubation times, the hydrolysed metabolites (Ala-Leu-doxorubicin, Leu-doxorubicin or doxorubicin) were extracted and quantified by HPLC analysis. The sum of the concentration (μM) of the three metabolites formed over a six hour time period is shown in FIG. 16. As previously observed in vitro, the pegylation (PEG_{20,000}) of the ALAL-Dox construct results in a significant decrease in reactivation and release of Ala-Leu-doxorubicin, Leu-doxorubicin and doxorubicin. These results demonstrate the importance of the (D-Serine)ₓ spacer in enhancing the peptidase specific cleavage of the Ala-Leu-Ala-Leu oligopeptide when conjugated to high molecular weight polymers such as PEG, which in turn can be used to improve the pharmacokinetic and tumor targeting properties of the prodrug.

### BIBLIOGRAPHIC REFERENCES

Boven, E.; Winograd, B.; Berger, D. P.; Dumont, M. P.; Braakhuis, B. J.; Fostad, O.; Langdon, S. and Fiebig, H. H., Cancer Res 1992, 52: 5940-5947.

Dubois, V.; Dasnois, L.; Lebtahi, K.; Collot, F.; Heylen, N.; Havaux, N.; Fernandez, A. M.; Lobl, T. J.; Oliyai, C.; Nieder, M.; Shochat, D.; Yarranton, G. T.; Trouet, A., Cancer Res. 2002 Apr. 15; 62(8):2327-31.

Duncan, R.; Spreafico, F., Clin. Pharmacokinetics 1994, 27 (4): 290-306.

Engel, L. W.; Young, N. A., Cancer Research 1978, 38: 4327-39.

Fernandez, A. M.; Van Derpoorten, K.; Dasnois, L.; Lebtahi, K.; Dubois, V.; Lobl, T.; Gangwar, S.; Oliyai, C.; Lewis, E. R.; Schochat, D.; and Trouet, A., J Med Chem 2001, 44:3750-3753

Greenwald, R. B.; Choe, Y. H.; McGuire, J.; Conover, C. D., Advanced Drug Delivery Reviews 2003, 55: 217-250.

Harris, J. M.; Chess, R. B., Effect of PEGylation on Pharmaceuticals. Nature Reviews, 2003, 2: 214-221.

Horoszewicz, J. S.; Leong, S. S.; Chu, T. M.; Wajsman, Z. L., Friedman, M., Papsidero, L.; Kim, U.; Chai, L. S.; Kakati, S.; Arya, S. K.; Sandberg, A. A., Prog Clin Biol Res. 1980; 37:115-32.

Kaiser E.; Colescott, R. L.; Bossinger, C. D.; and Cook, P. I., Anal. Biochem 1970, 34: 595-598.

Kyriazis, A. A., and Kyriazis, A. P., Cancer Res. 1980, 40: 4509-4511.

Langdon, S. P.; Hendriks, H. R.; Braakhuis, B. J.; Pratesi, G.; Berger, D. P.; Fodstad, O.; Fiebig, H. H. and Boven, E., Ann Oncol 1994, 5:415-422.

Merrifield, J., Amer. Chem. Soc. 1963, 85:2149-54.

Merrifield, J., Science 1965, 50:178-85.

Nicklas, W.; Baneux, P.; Boot, R.; Decelle, T.; Deeny, A. A.; Fumanelli, M., and Illgen-Wilcke, B., Lab Anim 2002, 36:20-42.

Rehbinder, C.; Basneux, P.; Forbes, D.; van Herck, H.; Nicklas, W.; Rugaya, Z.; and Winkler, G., FELASA Recommendations for the Health Monitoring of Mouse, Rat, Hamster, Gerbil, Guinea Pig and Rabbit Experimental Units. Report of the Federation of European Laboratory Animal Science Asoociations (FELASA) Working Group on Animal Health Accepted by FELASA Board of Management, November 1995. Lab Anim 1996, 30:193-208.

Rehbinder, C.; Alenius, S.; de las Heras, M. L.; Greko, C.; Kroon, P. S. and Gutzwiller, A., FELASA Recommendations for the Health Monitoring of Experimental Units of Claves, Sheep and Goats Report of the Federation of European Laboratory Animal Science Associations (FELASA) Working Group on Animal Health. Lab Anim 2000, 34: 329-350.

Seymour, L. W.; Miyamoto, Y.; Maeda, H.; Strohalm, J.; Ulbrich, K.; Duncan, R., Eur. J. Cancer 1995, 31:766-770.

Schmidt, F.; Ungureanu, I.; Duval, R.; Pompon, A.; Monneret, C., Eur. J. Org. Chem. 2001, 2129-2134.

Soule, H. D.; Vazguez, J.; Long, A.; Albert, S.; Brennan, M., J. Natl. Cancer Inst. 1973, 51(5): 1409-16.

Steward and Young, Solid Phase Peptide Synthesis 1969, W. H. Freeman & Co, San Francisco.

Trouet, A.; Passioukov, A.; Van derpoorten, K.; Fernandez, A. M.; Abarca-Quinones, J.; Baurain, R.; Lobl, T. J.; Oliyai, C.; Shochat, D.; Dubois, V., Cancer Res. 2001 1;61(7): 2843-6.

Workman, P.; Twentyman, P.; Balkwill, F.; Balmain, A.; Chaplin, D.; Double, J.; Embleton, J.; Newell, D.; Raymond, R.; Stables, J.; Stephens, T., and Wallace, J., United Kingdom Coordinating Committee on Cancer Research (UKCCCR) Guidelines for the Welfare of Animals in Experimental Neoplasia (Second Edition). Br J Cancer 1998, 77:1-10.

Virender, K.; Sarin, Stephen; B. H. Kent, James P. Tam, and Merrifield, R. B., Analytical Biochem. 1981, 117, 145-157.

What is claimed is:

1.) Compound of formula (A)ₚ-(E-B)ₙ-(I)ₘ, in which:
I is an active substance of interest against target cells,
B is a structure that can be cleaved selectively by an enzyme that is present only or preferably close to or at said target cells,
E is a hydrophilic spacer group, stable in the circulatory structure, which separates A from B so as to make possible or to facilitate the cleavage of B close to or at said target cells and thus to make possible or to facilitate the release of I or the release of I with a radical of B,
A is a group that increases the half-life time of B-I in the blood circulation,
E-B is a group connecting A and I, where:
n is an integer between 1 and either the total number of reactive functions of I on which connecting groups E-B can be coupled, or the total number of reactive functions of A on which connecting groups E-B can be coupled,
m is an integer between 1 and the total number of reactive functions of A, on which connecting groups E-B can be coupled, or the total number of reactive functions of B on which I can be coupled,
p is an integer between 1 and the total number of reactive functions of I, on which connecting groups E-B can be coupled, or the total number of reactive functions of E on which A can be coupled
and optionally wherein when p=1 then n=m, and when m=1 then n=p.

2.) A compound according to claim 1, comprising an I group that is attached directly to one or more structures B by a covalent bond.

3.) A compound according to claim 1, comprising an I group that is attached to one or more structures B via a connecting arm.

4.) A compound according to claim 1, comprising an E group wherein said group E comprises from about 1 to about 100 amino acids in size.

5.) A compound according to claim 1, comprising an E group wherein said group E comprises from about 1 to about 20 amino acids in size.

6.) A compound according to claim 1, wherein said group E comprises from about 2 to about 10 amino acids in size.

7.) A compound according to claim 1, wherein said group E comprises at least one amino acid that is a natural amino acid in the D conformation.

8.) A compound according to claim 1, comprising an E group wherein the amino acids of said group E are identical amino acids.

9.) A compound according to claim 1, comprising an E group wherein the amino acids of said group E are not genetically coded.

10.) A compound according to claim 7, wherein at least one of said amino acid is selected from: D-glutamine, D-asparagine, D-serine, D-histidine, D-threonine, D-aspartic acid, D-glutamic acid, D-lysine, D-arginine, and combinations thereof.

11.) A compound according to claim 1, comprising an E group wherein the amino acids of said E group comprise between about 1 to about 20 D-threonine amino acids.

12.) A compound according to claim 1, comprising an E group wherein the amino acids of said E group comprise between about 1 to about 20 D-serine amino acids.

13.) A compound according to claim 1, comprising an E group that is selected from a peptidomimetic agent, a pseudopeptide or a peptoid.

14.) A compound according to claim 1, wherein said E group comprises at least one group that is selected from: substituted alkyl chains, polyalkyl glycols, polysaccharides, polyols, polycarboxylates, or poly(hydro)esters.

15.) A compound according to claim 1, wherein the number of amino acids of said group E increases as the molecular weight of said group A becomes larger.

16.) A compound according to claim 1, comprising an A group further exhibiting the property of solubilization in water, blood or serum.

17.) A compound according to claim 1, comprising an A group further exhibiting targeting properties toward one or more target cells.

18.) A compound according to claim 1, comprising an A group that is an agent with therapeutic activity or diagnostic activity.

19.) A compound according to claim 1, comprising an A group that is hydrophilic or amphiphatic.

20.) A compound according to claim 1, comprising an A group that is selected from among: polypeptides, immunoglobulins, albumin, polysaccharides, polymers, or copolymers.

21.) A compound according to claim 20, comprising an A group that is selected from among: polyalkylene glycol, polyalkylene oxide, polyalkylene imine, or vinyl chloride copolymers.

22.) A compound according to claim 21, comprising an A group that is selected from among: a polyethylene glycol, a polyethylene oxide, a polyethylene imine, sodium styrene sulfonate (NaSS), sodium maleate and butyl maleate (MMBE), hydroxypropyl methacrylate or N-(2-hydroxypropyl)methacrylamide (HPMA), methyl methacrylate (MMA), poly-[N-(2-hydroxyethyl)-L-glutamine] (PHEG), poly-[N-(hydroxyethyl)-DL-aspartamide] (PHEA), or polylactic acid (PLA).

23.) A compound according to claim 21, comprising an A group that is a polyethylene glycol of a size between about 200 and about 50,000 Da.

24.) A compound according to claim 1, comprising a B group that is selectively cleaved by an enzyme that is present in the environment of one or more cells that are selected from the group of: tumor cells, stromal cells of tumors, neoangiogenic endothelial cells of tumors and tumor metastases, macrophages, monocytes, polymorphonuclear leukocytes or lymphocytes that infiltrate tumors and tumor metastases.

25.) A compound according to claim 1, comprising a B group that is selected from the group of: oligopeptides, oligosaccharides, or lipid chains.

26.) A compound according to claim 25, comprising a B group that is selected from the group of: L-alanine-L-leucine-L-alanine-L-leucine or L-alanine-L-tyrosine-L-glycine-L-glycine-L-phenylalanine-L-leucine.

27.) A compound according to claim 1, comprising a B group that is cleaved by an enzyme that is selected from the group of: peptidases, endopeptidases, lysosomal enzymes, lipases, or glycosidases.

28.) A compound according to claim 27, comprising a B group that is cleaved by a peptidase that is selectively present in the environment of the tumor cells, stromal cells of tumors, neoangiogenic endothelial cells, macrophages, monocytes, lymphocytes or polymorphonuclear leukocytes.

29.) A compound according to claim 28, wherein the peptidase of the tumor cells is selected from the group of: neprilysine (CD10), thimet oligopeptidase (TOP), prostate specific antigen (PSA), plasmine, legumaine, collagenases, urokinase, cathepsins, or the matrix metallopeptidases.

30.) A compound according to claim 1, comprising an I group that is selected from the group of: a chemical agent, a polypeptide, a protein, a nucleic acid, an antibiotic, or a virus or a marker, optionally coupled with a vector substance.

31.) A compound according to claim 30, comprising an I group that is an agent with anti-tumor therapeutic activity, anti-angiogenic activity or anti-inflammatory activity.

32.) A compound according to claim 31, comprising an I group that is selected from the group of: anthracyclines, doxorubicin, daunorubicin, folic acid derivatives, vinca alkaloids, calicheamicin, mitoxantrone, cytosine arabinoside, adenosine arabinoside, fludarabine phosphate, melphalan, bleomycins, mitomycins, L-canavanine, taxoids, camptothecin, 9-dimethylaminomethyl-hydroxy-camptothecin hydrochloride, proteasome inhibitors, farnesyl-transferase inhibitors (FTI), epothilones, maytansinoids, discodermolide, fostriecin, platinum derivatives, duocarmycins, combretastatin, epipodophyllotoxins, BH3 peptides, p53 peptides, caspases, granzyme B; ribozymes, tumor necrosis factor-alpha (TNF-alpha), interferon alpha (IFN-alpha), interferon gamma (IFN-gamma), interleukin 1 (IL-1), IL-2, IL-6, IL-12, IL-15, or IGF-1 antagonists.

33.) A compound according to claim 30, comprising an I group that is a marker selected from the group of: coumarin, 7-amido-trifluoromethyl coumarin, paranitroanilide, 8-naphthylamide and 4-methoxy naphthylamide, fluorosceine, biotin, rhodamine, and their derivatives, or the agents that are used in scintigraphy.

34.) A compound according to claim 1, further comprising one or more targeting substances that can vector said compound of formula (A)ₚ-(E-B)ₙ-(I)ₘ toward said target cells.

35.) A compound according to claim 34, wherein said one or more targeting substances are coupled with a compound of formula (A)ₚ-(E-B)ₙ-(I)ₘ at one or more of said A groups.

36.) A diagnostic or tracing pharmaceutical composition, further comprising as an active ingredient at least one compound according to any of claims 2, 3, 30, 31, 32 or 33.

37.) A pharmaceutical composition comprising the compound of claim 1.

38.) A compound comprising a polyethylene glycol substance selected from the group of:
a.) polyethylene glycol-DSeryl-DSeryl-DSeryl-DSeryl-LAlanyl-LLeucyl-LAlanyl-LLeucyl-doxorubicin;
b.) polyethylene glycol-DSeryl-DSeryl-DSeryl-DSeryl-LAlanyl-LLeucyl-LAlanyl-LLeucyl-TNFalpha;
c.) polyethylene glycol-DSeryl-DSeryl-DSeryl-DSeryl-LAlanyl-LTyrosyl-LGlycyl-LGlycyl-LPhenylalanyl-LLeucyl-doxorubicin; or
d.) polyethylene glycol-DSeryl-DSeryl-DSeryl-DSeryl-LAlanyl-LLeucyl-LAlanyl-LLeucyl-TNFalpha.
